SEQUENCE LISTING (7 sequences)

SEQ ID NO: 1 - DNA, 3356 bp, Arabidopsis thaliana
gtcgactcagctatgagattactcgtgtatcaattctctaattaattaaaaatagtataaattaaataatatagttcgatacacgaatataattgcgaagaataggcatacaaatttgtcatacatgtttcgatatggctcacgaggaggctgatgcaacagtttgatgtatacgtatgcaaattgagaagtacttgatcagacctatatatgtgatgctcgaacttatctttttgttttggatcatctatcgaatacaatggtactataatttaaatgttttttttcttctttttctttagtatcaaaagcaacgttagatgctaaataaagagttagttgattgtgatgactgatagtctgataatatcattaactttgcacccgaagtcaaataaaagtgttcatatttataaattccaaccaacgttaataagccacacctaatcggtgattgccaacaatattataataaaattaaaaaaactacgactaaagttaatttgctataattttgtggtatgttttaaaaataaagttctttagttctaatatcatgaaaattcagtgtactgtaaaatatgtaaaaaggttttagtacaattcttttttgtatataacggcaaagttcaatacatattttactattgattttttttaaaaataaaataacaattgctaccaactttttgaagcatattgatcgcaacttaattataattcttcttttttttcttggaagattaataaaacctaatttcaatgtggaacaaataaatgtagaaatattgttatcacaaactaatatatgatattttttaatattttcatatatacttttgagcttctgatgatataacagttttcattaaaatacaaattgtcgtgtactaatttttcttttgttcaagtatgtgataaaaatatgttgcaaaattgcgagttattataatggtacaaatatgtagagagaatacatgagaagagttaaaagaagcatgcttaagccaacagagagtggatccaaatgttgctttccagctttatacaaacgtatcacccacattactgccactgctacatatattgaaggagagagagatgatgatcataatgataaatcgatgtcgatgataaattgatgatgatggctccggtatgtgtaccctaggagttgtagctagctagctaggaccatgtatatacatacatacatatattagtgttttttgtaacttgtacgtaccttacaaacagtatggagtttactaaaacggcaacgtttggtgggggtagtgaattcgcaagtggggatgagtctatgtaatagaagatgcaacatgcaaatggtcccctttctgtttttatttaaagaaattagtgtttactgaggaggaaacatcccatttatagattcacacccataaaagcaaaccacttctccttctttttatttccccatgatattacttcgagaatattttgaaaatttgaagtgtacatttagagattgtgtactttgaacactcatgtcaaatgcatctaaatatataaactccaatttaaaataatcgtctaaacctagagtgccatttgtttagccatttgttggtcttcatttctcatgctttgattacatgtaccggttgattcatgtgaaaaatcatgtgcataaactaagaaatagctagcacataaaattttgatttaggttggatattactatgttcactttaagagaaaaaaaaacttatggcaaaaagtgatgatggtatatgaatatgataatcaaagtgcatatgtgaagtgagaggcaactgtagagtaatataataaaatccaaagaaaatttttaaatatgagaaaaaattatataaaaaggttcttttgtaatccacttcttttgatatagggagattcgttgagcatccatgtgctctttcaatcgacactattctgtctgtatctagccaacccacatatacctttacactagagaacttcgatgattctttttccaaaatcaatgtgatataatataattaagcatatatgcataaaaaatgaagaagaatggtagagtcatgttacttaaggtcatggtgtgtaaaaacattgatactttacaatatatgagttgtgaagtgctcttaaagttataacatccggttctacgtattgacctagaactagaagaatcgttttttagtccaaatcaaatcaagtcggttctttatcagttttgttgtatgtgaattaatttgaaaatattagctatgatcttagcttgggtttttgtttctaagggttaaggatcatatctctttgtcaaatgacatgtggtctatatgtcatgaattaggcaccgctatcttttactattgattcgacgacattgggactcctcactacacttatcttaaaaaaactcaaagttggtgttaatggcttgtcaccataaactttcatgagctctaacaaattaaacttgaacttgatcaggtctcacaatatatacaatttcgagggataaatatttcaaaaggataatatgatagttggtagaaatgtatagtttctagtaataatagagatcgttggttaaactccccaactttttaaaattaatttgattagtggatccgcaaacaaatattagattgggcctatatgcatctatattatttttatttttctgtaatttcagtaaaatgggcctatggtcctatatgcatccgaataattagtatactgggcttatgggcctatatgcatttgattttatcgataaaatgtgagtcaaatgtctaatgtgcgccgttatgaagtgcaagtggctaatttttttcacctagattccttctattgaccgtcgatagacggatgataactatgacgtggcattatcgcagccatcaaacaaagtcatgtataacaaacaagagcacacaaacgaaaacaaattcagttgcggaacccaaattcaaatcaacggaattagaatcacgctttcaattccgtaacccgccattaaaaaccttgaaccctcgaagcaaatcgagcaaagattttcaaatttcgaatttcaaaattctatctctctcactcttccaagcttagagagtcttagagcgagaaatctagagcttctcttaagtaagtgattgatctctctctttctctctactacgattcttcttcttcttctccattcatcgttttggtttaagctttgtcttaagttttgtgtacctgactcgcttcttctcgtttttattttgttttccgatgatcctgatctgtttgtgttgtttcggattcatagagctgaagaaacgagatctctcgagcccggg

SEQ ID NO: 2 - DNA, 1790 bp, Arabidopsis thaliana
gtcgacgaattcgcaagtggggatgagtctatgtaatagaagatgcaacatgcaaatggtcccctttctgtttttatttaaagaaattagtgtttactgaggaggaaacatcccatttatagattcacacccataaaagcaaaccacttctccttctttttatttccccatgatattacttcgagaatattttgaaaatttgaagtgtacatttagagattgtgtactttgaacactcatgtcaaatgcatctaaatatataaactccaatttaaaataatcgtctaaacctagagtgccatttgtttagccatttgttggtcttcatttctcatgctttgattacatgtaccggttgattcatgtgaaaaatcatgtgcataaactaagaaatagctagcacataaaattttgatttaggttggatattactatgttcactttaagagaaaaaaaaacttatggcaaaaagtgatgatggtatatgaatatgataatcaaagtgcatatgtgaagtgagaggcaactgtagagtaatataataaaatccaaagaaaatttttaaatatgagaaaaaattatataaaaaggttcttttgtaatccacttcttttgatatagggagattcgttgagcatccatgtgctctttcaatcgacactattctgtctgtatctagccaacccacatatacctttacactagagaacttcgatgattctttttccaaaatcaatgtgatataatataattaagcatatatgcataaaaaatgaagaagaatggtagagtcatgttacttaaggtcatggtgtgtaaaaacattgatactttacaatatatgagttgtgaagtgctcttaaagttataacatccggttctacgtattgacctagaactagaagaatcgttttttagtccaaatcaaatcaagtcggttctttatcagttttgttgtatgtgaattaatttgaaaatattagctatgatcttagcttgggtttttgtttctaagggttaaggatcatatctctttgtcaaatgacatgtggtctatatgtcatgaattaggcaccgctatcttttactattgattcgacgacattgggactcctcactacacttatcttaaaaaaactcaaagttggtgttaatggcttgtcaccataaactttcatgagctctaacaaattaaacttgaacttgatcaggtctcacaatatatacaatttcgagggataaatatttcaaaaggataatatgatagttggtagaaatgtatagtttctagtaataatagagatcgttggttaaactccccaactttttaaaattaatttgattagtggatccgcaaacaaatattagattgggcctatatgcatctatattatttttatttttctgtaatttcagtaaaatgggcctatggtcctatatgcatccgaataattagtatactgggcttatgggcctatatgcatttgattttatcgataaaatgtgagtcaaatgtctaatgtgcgccgttatgaagtgcaagtggctaatttttttcacctagattccttctattgaccgtcgatagacggatgataactatgacgtggcattatcgcagccatcaaacaaagtcatgtataacaaacaagagcacacaaacgaaaacaaattcagttgcggaacccaaattcaaatcaacggaattagaatcacgctttcaattccgtaacccgccattaaaaaccttgaaccctcgaagcaaatcggtacccgggagatctccatgg

SEQ ID NO: 3 - protein, 339 aa, Human
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 4 - protein, 342 aa, Lycopersicon esculentum
MEQQHRNQKSMQDQNDEIEDVQHGPFPVEQLQASGIAALDVKKLKDAGLCTVESVVYAPRKELLQIKGISEAKVDKIIEAASKLVPLGFTSASQLHAQRLEIIQITSGSKELDKILEGGIETGSITEIYGEFRCGKTQLCHTLCVTCQLPLDQGGGEGKAMYIDAEGTFRPQRLLQIADRYGLNGPDVLENVAYARAYNTDHQSRLLLEAASMMVETRFALMIVDSATALYRTDFSGRGELSARQMHLAKFLRSLQKLADEFGVAVVITNQVVAQVDGSAVFAGPQIKPIGGNIMAHASTTRLALRKGRAEERICKVVSSPCLAEAEARFQISVEGVTDVKD

SEQ ID NO: 5 - protein, 340 aa, Zea mays (zmrad51a)
MSSAAQQQQKAAAAEQEEVEHGPFPIEQLQASGIAALDVKKLKDSGLHTVEAVAYTPRKDLLQIKGISEAKADKIIEAASKIVPLGFTSASQLHAQRLEIIQVTTGSRELDKILEGGIETGSITEIYGEFRSGKTQLCHTPCVTCQLPLDQGGGEGKALYIDAEGTFRPQRLLQIADRFGLNGADVLENVAYARAYNTDHQSRLLLEAASMMIETRFALMVVDSATALYRTDFSGRGELSARQMHMAKFLRSLQKLADEFGVAVVITNQVVAQVDGSAMFAGPQFKPIGGNIMAHASTTRLALRKGRGEERICKVISSPCLAEAEARFQLASEGIADVKD

SEQ ID NO: 6 - protein, 340 aa, Zea mays (zmrad51b)
MSSSSAHQKASPPIEEEATEHGPFPIEQLQASGIAALDVKKLKDAGLCTVESVAYSPRKDLLQIKGISEAKVDKIIEAASKLVPLGFTSASQLHAQRLEIIQLTTGSRELDQILDGGIETGSITEMYGEFRSGKTQLCHTLCVTCQLPLDQGGGEGKALYIDAEGTFRPQRILQIADRFGLNGADVLENVAYARAYNTDHQSRLLLEAASMMVETRFALMVVDSATALYRTDFSGRGELSARQMHLAKFLRSLQKLADEFGVAVVITNQVVAQVDGAAMFAGPQIKPIGGNIMAHASTTRLFLRKGRGEERICKVISSPCLAEAEARFQISSEGVTDVKD

SEQ ID NO: 7 - protein, 342 aa, Arabidopsis thaliana
MTTMEQRRNQNAVQQQDDEETQHGPFPVEQLQAAGIASVDVKKLRDAGLCTVEGVAYTPRKDLLQIKGISDAKVDKIVEAASKLVPLGFTSASQLHAQRQEIIQITSGSRELDKVLEGGIETGSITELYGEFRSGKTQLCHTLCVTCQLPMDQGGGEGKAMYIDAEGTFRPQRLLQIADRFGLNGADVLENVAYARAYNTDHQSRLLLEAASMMIETRFALLIVDSATALYRTDFSGRGELSARQMHLAKFLRSLQKLADEFGVAVVITNQVVAQVDGSALFAGPQFKPIGGNIMAHATTTRLALRKGRAEERICKVISSPCLPEAEARFQISTEGVTDCKD

(19) United States
(12) Patent Application Publication
(10) Pub. No.: US 2004/0111764 A1
(43) Pub. Date: Jun. 10, 2004

(54) Composition and method for increased meiotic recombination in plants

(76) Inventors: Andreas Stefan Betzner, Page (AU); Eric Huttner, Aranda (AU); Pascual Perez, Chanonat (FR); Marie-Pascale Doutriaux, Saulx Les Chartreux (FR); Charles White, Perignat Les Sarlieve (FR)

Correspondence Address: Connolly Bove Lodge & Hutz, P O Box 2207, Wilmington, DE, 19899-2207, US

(21) Appl. No.: 10/333,806

(22) PCT Filed: Jul. 25, 2001
(86) PCT No.: PCT/EP01/10185

(30) Foreign Application Priority Data
Jul. 26, 2000 (AU) PQ 9015

(51) Int. Cl. A01H 1/00; C12N 15/82

(52) U.S. Cl. 800/287; 435/468; 435/320.1

(57) ABSTRACT

The present invention provides an expression cassette comprising a meiotically active promoter operably linked to a polynucleotide encoding a recombinational DNA repair polypeptide, or fragment thereof, wherein said polynucleotide is capable of stimulating plant meiotic recombination when expressed into RNA and/or said polypeptide.

### TECHNICAL FIELD

The present invention relates to meiosis in plants, and more specifically, increasing the frequency of meiotic recombination in plants, via the insertion of a genetic construct, the construct comprising a meiotically active promoter operably linked to a polynucleotide encoding a recombinational DNA repair polypeptide, or fragment thereof, wherein said polynucleotide is capable of stimulating plant meiotic recombination when expressed into RNA and/or said polypeptide.

### BACKGROUND ART

Plant breeding is a slow and unpredictable process, traditionally relying on chromosome partitioning and recombination at meiosis to produce genetic variation as the basis for selection and development of germplasm. Random partitioning of parental chromosomes to the gametes allows for new combination of unlinked traits, which may segregate in subsequent generations. Meiotic recombination, on the other hand, exchanges genetic information between homologous or homeologous parental chromosomes. As a result, stable new linkages can be formed in-between traits, and old ones can be broken.

Both formation and breakage of genetic linkages can be useful in plant breeding. The formation of linkages allows the addition of desired genetic information from agronomically less significant genomes to homologous or homeologous chromosomes of agronomically important genomes. Non-limiting examples are the introgression into crops, or into elite lines of crops, of natural pathogen resistance genes from wild relatives, and of transgenes from transformation-competent but otherwise inferior crop lines. Recombination will also be helpful to introgression of apomixis into crops, such as the transfer of diplospory from Tripsacum to Zea mais. On the other hand, the breakage of linkages allows the separation of undesired traits from otherwise preferred linkage groups. The latter strategy can be employed to separate for example the undesirable locus for glucosinolate production from the desirable and closely linked Rfo locus, that restores fertility to the Ogura cytoplasmic sterility in Brassica napus.

In contrast to parental chromosome partitioning, meiotic recombination is a non-random process and therefore is potentially amenable to manipulation by genetic engineering. Such manipulation may be intended to increase a normally low or very low recombination frequency between two loci that are in close proximity to each other, or are located in or near regions of recombination inactive chromatin. In such, and other cases, increased meiotic recombination can significantly shorten the length of breeding programs, as it allows a reduction in the number of parental crosses and the size of F2 screening populations usually required to identify a rare or very rare recombination event at F1 meiosis.

Accordingly, there is a need for methods to increase the frequency of recombination in plants, thereby improving the efficiency of plant breeding processes.

Numerous genes are known to affect recombination at different levels in yeast [for review see N. Kleckner (1996), PNAS 93, 8167-8174; K. N. Smith and A. Nicolas (1998), Curr. Opin. Genet. Dev. 8, 200-211; A. Shinohara and T. Ogawa (1999) Mutation Research 435, 13-21]. Some of these genes were subsequently isolated also from animals, and their gene products can be divided into three functional groups.

The first group comprises those proteins that execute recombination, such as RAD51 and DMC1. Of theses only DMC1 is specific to meiosis, while RAD51 participates in both somatic and meiotic recombination. Accordingly, the two proteins differ in their substrate preference, that is, DMC1 is involved predominantly in interhomologue recombination whereas RAD51 acts preferentially in recombination between the sister chromatids of a chromosome [for review see J. E. Haber (2000), Trends in Genetics 16, 259-264)]. Both proteins share a high similarity to each other and to the bacterial RecA protein with respect to amino acid sequence and to protein function [A. Shinohara et al. (1992), Cell 69, 457-470; D. K Bishop et al. (1992), Cell 69, 439-456; A. I. Roca and M. M. Cox (1997), Prog. Nucleic Acid Res. Mol. Biol. 56, 129-223].

The second group consists of proteins which support recombination by formation of meiotic double-strand breaks (eg. SPO11, MER1, MER2, MRE2, MEI4, REC102, REC104, REC114, RAD50, MRE11, XRS2), and by providing access to chromatin (eg. RAD18/SMC, and others) [A. R. Lehmann et al. (1995), Mol. Cell. Biol. 15, 7067-7080]. Other proteins within this second group are involved in the processing of double stand breaks or otherwise assist in DNA strand exchange (eg. RAD52, RAD54, RDH54/TID1, RAD55-57, and others), whereby some of them (eg. RAD54, RDH54/TID1, RAD55) directly interact with RAD51 protein and with DMC1 protein respectively. [J. E. Haber (1997), Trends in Genetics 16, 259-264; M. Shinohara et al. (1997), Genetics 147, 1545-1556; A. Shinohara and T. Ogawa (1999) Mutation Research 435, 13-21; P. Uetz et al. (2000), Nature 403, 623-627]. However, not all of these “group 2” proteins are specific to meiotic recombination. In fact, most RAD proteins, as well as MRE11 and XRS2, are also involved in somatic recombination which is mechanistically similar to meiotic recombination since both can be described with the double-strand DNA break repair model [J. W. Szostak et al. (1983), Cell 33, 25-35; K. N. Smith and A. Nicolas (1998), Curr. Opin. Genet. Dev. 8, 200-211].

Finally, a third group of proteins include those having functions which normally hinder meiotic recombination (eg. mismatch repair functions), or control it.

Whilst methods to promote recombination in somatic cells have been described in U.S. Pat. No. 5,945,339, U.S. Pat. No. 5,780,296, and Vispe et al. (1998) NAR 26: 2859-2864, these methods have primarily been developed for gene targeting, and have not been applied to plant cells, or whole multi-cellular organisms, such as plants. Moreover, these methods relate to mitosis and somatic cells, and not to meiosis, or to meiotic cells.

Although meiotic recombination is highly relevant to plant breeding, there is very little knowledge concerning the function of genes involved in meiotic recombination. Recently, homologues of yeast RAD18 (SMC), DMC1 and RAD51 have been isolated from plants [T. Kobayashi et al. (1993), Mol. Gen. Genet 237, 225-232; V. I. Klimyuk and J. D. G. Jones (1997), Plant J. 11, 1-14; M. P. Doutriaux et al. (1998), Mol. Gen. Genet 257, 283-291; T. Mengiste et al. (1999), EMBO J. 18, 4505-4512; A. E. Franklin et al. (1999), Plant Cell 11, 809-824].

Of these, the SMC-like protein MIM of Arabidopsis (a putative homologue to yeast RAD18) was proposed to contribute to DNA damage repair and thus to intragenic recombination in plant cells, but a potential meiotic function was not demonstrated [T. Mengiste et al. (1999), EMBO J. 18, 4505-4512; WO 00/04174]. As for Arabidopsis DMC1, meiotic function during chromosome pairing and/or recombination was shown by mutant analysis, and its promoter was proposed to be useful as a tool for transgene expression in meiotic plant cells [WO 98/284331]. However, in contrast to yeast DMC1, the Arabidopsis homologue was also found to be expressed in somatic cells (suspension culture), and its inactivation at meiosis did not lead to meiotic cell cycle arrest [M. P. Doutriaux et al. (1998), Mol. Gen. Genet 257, 283-291; F. Couteau et al. (1999), Plant Cell 11, 1623-1634]. These rather unexpected findings with Arabidopsis DMC1 indicate that, despite all similarities, differences do exist between eukaryotic organisms, with respect to control and execution of meiotic prophase and to individual protein function. Such reservations may also apply to genes encoding recombinationally active proteins, such as the polynucleotide encoding plant RAD51, because suitable knock-out mutants from which the RAD51 function could be deduced are not yet available in any plant.

Therefore, whilst genetic engineering of recombination at meiosis holds promise for use in plant breeding techniques, at present the knowledge of meiosis in plants remains limited and suitable methods for increasing meiotic recombination have not been described which would make use of recombinational DNA repair functions.

The present invention however, provides the means for increasing meiotic recombination in plants. The invention stems from the combination of a meiotically active promoter operably linked to a polynucleotide encoding a recombinational DNA repair polypeptide, or fragment thereof, wherein said polynucleotide is capable of stimulating plant meiotic recombination when expressed into RNA and/or said polypeptide. The present invention is further directed to vectors, host cells, transgenic plants and methods for increasing recombination at meiosis in plants in order to improve plant breeding.

### DISCLOSURE OF THE INVENTION

The present invention relates to the improvement of plant breeding processes, and compositions and methods for use therein are provided. In one form, the invention describes compositions, such as expression cassettes, comprising meiotically active promoters operably linked to a polynucleotide that encodes a protein involved in recombinational DNA repair, wherein said polynucleotide is capable of stimulating meiotic recombination in plants when expressed into RNA and/or protein. In another form, the present invention describes a method for elevating the frequency of meiotic recombination in plants comprising expressing these polynucleotides or expression cassettes, respectively.

In a preferred aspect, the present invention relates to increased meiotic recombination in plants resulting from meiotic expression of a polynucleotide encoding RAD51. It also relates to use of meiotically active plant promoters in order to restrict as much as possible the expression of introduced DNA repair functions to the meiocytes of a transgenic plant. Generally, the method for elevating the frequency of meiotic recombination in plants is by deregulation of DNA repair functions during zygotene and pachytene of meiosis I, that is, during chromosome synapsis and recombination.

According to a first embodiment of the invention, there is provided an expression cassette comprising a meiotically active promoter operably linked to a polynucleotide encoding a recombinational DNA repair polypeptide, or fragment thereof, wherein said polynucleotide is capable of stimulating plant meiotic recombination when expressed into RNA and/or said polypeptide.

Typically, the polynucleotide capable of stimulating meiotic recombination in plants encodes a recombinational DNA repair polypeptide, or a fragment thereof, selected from the group consisting of: SPO11 (protein ID AAA65532.1), MER1 (protein ID NP_{—}014189), MER2 (protein ID AAA34772.1), MRE2 (protein ID BAA02016.1), MEI4 (protein ID NP_{—}010963.1), REC102 (protein ID AAA34964.1), REC104 (protein ID AAB26085.1), REC114 (protein ID NP_{—}013852.1), MRE11 (protein ID BAA02017.1), XRS2 (protein ID AAA35220.1), RAD18 (SMC) (protein ID AAA34932.1), RAD50 (protein ID CAA32919.1), RAD51 (protein ID BAA00913.1, protein ID CAA45563, protein ID AAB37762.1, protein ID AAD32030.1, protein ID AAD32029.1, AAC23700 or AAF69145.1), RAD52 (protein ID AAA50352.1), RAD54 (protein ID AAA34949.1), RDH54/TID1 (protein ID NP_{—}009629), RAD55-57 (protein ID protein m AAA19688.1, protein ID AAA34950.1), DMC1 (protein ID NP_{—}011106.1), and Arabidopsis protein XRS9, or functional fragments or analogues thereof (wherein the protein ID provides a cross-reference to GenBank for the corresponding nucleic acid sequence encoding the relevant polypeptide).

More typically, the polynucleotide encodes a RAD51 polypeptide, or a fragment thereof. Even more typically, the polypeptide is a plant RAD51 polypeptide, or a fragment thereof. Still more typically, the polypeptide is the Arabidopsis thaliana RAD51 represented by protein ID AAB37762.1 (AtRAD51), or a fragment thereof. Yet more typically, the polypeptide is the Zea mais RAD51 represented by protein ID AAD32029.1 (ZmRAD51A), or protein ID AAD32030.1 (ZmRAD:51B), or fragments thereof. Even more typically, the polypeptide is the tomato RAD51 polypeptide represented by protein ID No AAC23700 (LeRAD51) or a fragment thereof. In relation to this, the protein ID provides a cross-reference to GenBank for the corresponding nucleic acid sequence encoding the relevant polypeptide.

More typically, the polynucleotide encodes the N-terminal domain of a polypeptide corresponding to the human RAD51 (protein ID AAF69145.1): amino acid position 1-114, or the C-terminal domain corresponding to the human RAD51: amino acid position 115-339, and this is further outlined in Example 1 and FIG. 1B below.

Typically, the meiotically active promoter defined in the first embodiment of the invention is a meiosis specific promoter. Even more typically, the promoter is a plant meiosis specific promoter. Still more typically, the promoter is active during zygotene and pachytene of meiosis I in plants. Even still more typically, the promoter is a plant DMC1 promoter, wherein DMC1 is described in V. I. Klimyuk and J. D. G. Jones, 1997, Plant J. 11, 1-14 and its nucleic acid sequence is provided in GenBank Accession No. U76670, the disclosure of which is incorporated herein by reference. Still more typically, the promoter is the polynucleotide of DMC1 short, or fragment thereof, and the corresponding sequence with modifications is outlined in SEQ ID NO:1. Yet still more typically, the promoter is the polynucleotide of DMC1 long, or fragment thereof, and the corresponding sequence with modifications is outlined in SEQ ID NO: 1.

Generally, the meiotically active promoter is of weak to medium strength, and reference is made to Example 4 in this respect. Typically, the meiotically active promoter is as strong as DMC1 long outlined above, or is weaker.

Typically, the RNA defined in accordance with the first embodiment of the invention is present as mRNA, or fragments thereof. More typically, it is capable of stimulating plant meiotic recombination and encodes the recombinational DNA repair polypeptide, or fragment thereof, in either sense or anti-sense orientation, with respect to the meiotically active promoter.

Typically, the polynucleotides of the first embodiment of the invention, or those polynucleotides encoding the promoters outlined above, also include within their scope analogues of the nucleic acid sequences defined above. Further, these analogue polynucleotides can be located and isolated using standard techniques in molecular biology, without undue trial and experimentation.

Typically, the nucleic acid molecules include within their scope analogues which have at least 60% homology to the polynucleotide sequences so defined. More typically, the analogues of the nucleic acid molecules have at least 70% homology, still more typically the analogues have at least 75% homology, even more typically, the analogues have at least 80% homology, still more typically, the analogues have at least 85% homology, and yet still more typically, the analogees have at least 90% homology, and yet even still more typically, the analogues have at least 95-99% homology to the nucleic acid molecules defined above.

The degree of homology between two nucleic acid sequences may be determined by means of computer programs known in the art such as GAP provided in the GCG program package (Program Manual for the wisconsin Package, Version 8, August 1996, Genetics Computer Group, 575 Science Drive, Madison, Wis., USA 53711) (Needleman, S. B. and Wunsch, C. D., (1970), Journal of Molecular Biology, 48, 443-453). Using GAP with the following settings for DNA sequence comparison: GAP creation penalty of 5.0 and GAP extension penalty of 0.3.

Nucleic acid molecules may be aligned to each other using the Pileup alignment software, available as part of the GCG program package, using, for instance, the default settings of gap creation penalty of 5 and gap width penalty of 0.3.

Typically, the nucleic acid molecules also includes within their scope analogues capable of hybridising to the nucleic acid molecules defined above under conditions of low stringency, wherein low stringency hybridisation conditions typically correspond to hybridisation performed at 40 to 50° C. in 4 to 6×SSC. More typically, analogues capable of hybridising to the nucleic acid molecules defined above are identified under conditions of medium stringency, wherein medium stringency hybridisation conditions typically correspond to hybridisation performed at 55 to 60° C. in 0.5 to 1×SSC. Even more typically, analogues capable of hybridising to the nucleic acid molecules defined above are identified under conditions of high stringency, wherein high stringency hybridisation conditions typically correspond to hybridisation performed at 60 to 65° C. in 0.1 to 0.5×SSC.

In general, suitable experimental conditions for determining whether a given nucleic acid molecule hybridises to a specified nucleic acid may involve presoaking of a filter containing a relevant sample of the nucleic acid to be examined in 5×SSC for 10 min, and prehybridisation of the filter in a solution of 5×SSC, 5×Denhardt's solution, 0.5% SDS and 100 μg/ml of denatured sonicated salmon sperm DNA, followed by hybridisation in the same solution containing a concentration of 10 ng/ml of a ³²PdCTP-labeled probe for 12 hours at approximately 45° C., in accordance with the hybridisation methods as described in Sambrook et al. (1989; Molecular Cloning, A Laboratory Manual, 2nd edition, Cold Spring Harbour, N.Y.).

The filter is then washed twice for 30 minutes in 2×SSC, 0.5% SDS at least 55° C. (low stringency wash), at least 60° C. (medium stringency wash), at least 65° C. (medium/high stringency wash), at least 70° C. (high stringency wash), or at least 75° C. (very high stringency wash). Hybridisation may be detected by exposure of the filter to an x-ray film or a phosphorimager cassette.

Also, there are numerous conditions and factors, well known to those skilled in the art, which may be employed to alter the stringency of hybridisation. For instance, the length and nature of the nucleic acid to be hybridised to the specified nucleic acid; concentration of salt and other components (such as the presence or absence of formamide, dextran sulfate, polyethylene glycol etc); and altering the temperature of the hybridisation and/or washing steps.

Further, it is possible to theoretically predict whether or not two given nucleic acid sequences will hybridise under certain specified conditions. Accordingly, as an alternative to the empirical method described above, the determination as to whether an analogous nucleic acid sequence will hybridise to the nucleic acid molecule defined above can be based on a theoretical calculation of the Tₘ (melting temperature) at which two heterologous nucleic acid sequences with known sequences will hybridise under specified conditions, such as salt concentration and temperature.

In determining the melting temperature for heterologous nucleic acid sequences (Tₘ₍ₕₑₜₑᵣₒ₎) it is necessary first to determine the melting temperature Tₘ₍ₕₒₘₒ₎) for homologous nucleic acid sequence. The melting temperature (Tₘ₍ₕₒₘₒ₎) between two fully complementary nucleic acid strands (homoduplex formation) may be determined in accordance with the following formula, as outlined in Current Protocols in Molecular Biology, John Wiley and Sons, 1995, as:

Tₘ₍ₕₒₘₒ₎=81.5° C.+16.6(log M)+0.41(% GC)−0.61(% form)−500/L

M=denotes the molarity of monovalent cations,

% GC=% guanine (G) and cytosine (C) of total number of bases in the sequence,

% form=% formamide in the hybridisation buffer, and

L=the length of the nucleic acid sequence.

Tₘ determined by the above formula is the Tₘ of a homoduplex formation (Tₘ₍ₕₒₘₒ₎) between two fully complementary nucleic acid sequences. In order to adapt the Tₘ value to that of two heterologous nucleic acid sequences, it is assumed that a 1% difference in nucleotide sequence between two heterologous sequences equals a 1° C. decrease in Tₘ. Therefore, the Tₘ₍ₕₑₜₑᵣₒ₎ for the heteroduplex formation is obtained through subtracting the homology % difference between the analogous sequence in question and the nucleotide probe described above from the Tₘ₍ₕₒₘₒ₎.

Typically the nucleic acid molecules also include within their scope functional fragments thereof. More typically, the fragment of the nucleic acid is an oligonucleotide fragment thereof. Typically, the oligonucleotide fragment is between about 15 to about 1100 nucleotides in length. More typically, the oligonucleotide fragment is between about 15 to about 680 nucleotides in length. Even more typically, the oligonucleotide fragment is between about 15 to about 350 nucleotides in length. Even more typically still, the oligonucleotide fragment is between about 15 to about 90 nucleotides in length. Yet still more typically, the oligonucleotide fragment is between about 15 to about 60 nucleotides in length.

According to a second embodiment of the invention, there is provided a recombinant vector comprising the expression cassette in accordance with the first embodiment of the invention.

Typically, the vector includes expression control sequences, such as an origin of replication for vector maintenance in bacteria, yeasts or plants, a promoter, an enhancer, and necessary processing information sites, such as ribosome binding sites, RNA splice sites, polyadenylation sites, and transcriptional terminator sequences. Still more typically, the vector may include one or more selection markers to permit detection of those cells transformed with the desired polynucleotide sequences. Examples of such selection markers include genes which confer phenotypic traits such as antibiotic, herbicide or disease resistance, or some other recognisable trait such as grain size, grain colour, growth rate, flowering time, ripening time etc.

Typically, the vector may be a cloning vector. More typically, such a cloning vector contains the bacterial replicon of ColE1, pMB1, p15A, pSC101, or pR6, or that of Ti or Ri plasmids. Still more typically, the vector may include the expression cassette between the right and left borders of a T-DNA which is derived from a tumor inducing (Ti) or from a root inducing (Ri) plasmid. Still more typically, the vector may further include at least one selection marker between the right and left borders of the T-DNA.

Commonly used plant transformation vectors useful in the present invention include for example: pBIN19 (Bevan et al., 1994, Nucleic Acids Research 12, 8711) and modifications thereof, or pGA492 (G. An et al., 1986, Plant Physiology 81, 86-91) and modifications thereof, or cosmid vectors such as pOCA18 (Nucleic Acids R search 16, 10765-10782) and pCIT (H. Ma et al., 1992, Gene 117, 161-167) and derivatives thereof, or bacterial artificial chromosomes (C. M. Hamilton, 1997, Gene 200(1-2):107-116). Still other typical vectors may be derived from plant DNA viruses or plant RNA viruses.

Typically, the vector may include heterologous coding sequence or sequences to permit the expression of transcriptional and translational fusions encoding the nucleic acid molecule of the invention, under the control of the meiotically active promoters outlined above.

According to a third embodiment of the invention, there is provided a host cell transformed with the expression cassette in accordance with the first embodiment of the invention, or the vector in accordance with the second embodiment of the invention.

Typically, the host cell is a plant cell. More typically, the host cell is a plant cell selected from any one of the following tissues: leaf, root, seed, stem or flower tissues. Even more typically, the host cell is a cell of a monocotyledenous or dicotyledenous plant. Still more typically, the host cell is a plant cell selected from the group of plants consisting of members of the following families: Cruciferae, Umbelliferae, Gramineae, Solanaceae, Compositae, Malvaceae, Leguminosae and Cucurbitaceae. Yet still more typically, the host cell is a plant cell selected from the following crops of these families consisting of oil seed rape, cauliflower and broccoli (Cruciferae); carrot (Umbelliferae); maize, wheat and barley (Gramineae); tomato, potato and tobacco (Solanaceae); sunflower (Compositae); cotton (Malvaceae); soybean and pea (Leguminosae); and melon (Cucurbitacea).

According to a fourth embodiment of the invention, there is provided a plant comprising the host cell as defined in accordance with the third embodiment of the invention.

Typically, the plant in accordance with the fourth embodiment of the invention is regenerated from the host cell defined in accordance with the third embodiment of the invention.

According to a fifth embodiment of the invention, there is provided a plant transformed or transfected with the expression cassette in accordance with the first embodiment of the invention, or the vector as defined in accordance with the second embodiment of the invention.

Typically, the plant as defined in accordance with the fourth or fifth embodiments of the invention is a monocotyledenous or dicotyledenous plant. More typically, the plant is selected from the group of plants consisting of members of the following families: Cruciferae, Umbelliferae, Gramineae, Solanaceae, Compositae, Malvaceae, Leguminosae and Cucurbitaceae. Even more typically, the plant is selected from the following crops of these families consisting of oil seed rape, cauliflower and broccoli (Cruciferae); carrot (Umbelliferae); maize, wheat and barley (Gramineae); tomato, potato and tobacco (Solanaceae); sunflower (Compositae); cotton (Malvaceae); soybean and pea (Leguminosae); and melon (Cucurbitaceae).

According to a sixth embodiment of the invention, there is provided seed of the plant defined in accordance with the fourth or fifth embodiments of the invention.

According to a seventh embodiment of the invention, there is provided a method for increasing the frequency of homologous or homeologous recombination in a plant, wherein said method comprises

a) transforming or transfecting a plant cell or tissue with a polynucleotide encoding a recombinational DNA repair polypeptide, or a fragment thereof, wherein said polynucleotide is capable of stimulating plant meiotic recombination when expressed into RNA and/or said polypeptide, or said polynucleotide is capable of stimulating plant meiotic recombination when introduced into said plant cell or tissue as an RNA::DNA chimeric molecule;

b) culturing said transformed or transfected plant cell or tissue under conditions allowing the regeneration of a plant,

c) culturing said regenerated plant under conditions allowing sexual reproduction of said regenerated plant; and

d) expressing said polynucleotide in said regenerated plant;

e) obtaining a sexually reproduced plant which is the product of said sexual reproduction; and

f) screening said sexually reproduced plant and/or its progeny for homologous or homeologous recombination events.

According to an eighth embodiment of the invention, there is provided a method for increasing the frequency of homologous or homeologous recombination in a plant, wherein said method comprises:

a) transforming or transfecting a plant cell or tissue with an expression cassette in accordance with the first embodiment of the invention, or the vector in accordance with the second embodiment of the invention;

b) culturing said transformed or transfected plant cell or tissue under conditions allowing the regeneration of a plant,

c) culturing said regenerated plant under conditions allowing sexual reproduction of said regenerated plant; and

d) expressing said polynucleotide in said regenerated plant;

e) obtaining a sexually reproduced plant which is the product of said sexual reproduction; and

f) screening said sexually reproduced plant and/or its progeny for homologous or homeologous recombination events.

Typically, the plant regenerated from the transformed or transfected plant cell or tissue in the course of the method defined in the seventh or eighth embodiments of the invention, is crossed with a plant from a second plant line, to generate a hybrid plant, such that the hybrid plant represents the combination of the genomes of at east two parent plants. The polynucleotide encoding the recombinational DNA repair polypeptide of the invention is then expressed in cells capable of undergoing meiosis in the hybrid plant line. The hybrid plant line is then permitted to sexually reproduce (preferably by self-fertilisation), and recombination events are identified in the resulting progeny.

Alternatively, the plant cell or tissue transformed or transfected in the course of the method defined in the seventh or eighth embodiments of the invention may be obtained from a hybrid plant, wherein the hybrid plant is itself derived from crossing a plant from a first parent line with a plant from a second parent line, such that the hybrid plant represents the combination of the genomes of at least two parent plants, between which recombination events are to be generated. In this manner, recombination events may be identified in the regenerated plant and/or the progeny of said plant.

According to an ninth embodiment of the invention, there is provided a method for increasing the frequency of homologous or homeologous meiotic recombination in a plant cell capable of undergoing meiosis, wherein said method comprises transforming or transfecting said plant cell with a polynucleotide encoding a recombinational DNA repair polypeptide, or a fragment thereof, wherein said polynucleotide is capable of stimulating meiotic recombination when expressed into RNA and/or said polypeptide, or said polynucleotide is capable of stimulating meiotic recombination when introduced into said plant cell as an RNA::DNA chimeric molecule.

According to a tenth embodiment of the invention, there is provided a method for increasing the frequency of homologous or homeologous meiotic recombination in a plant cell capable of undergoing meiosis, wherein said method comprises transforming or transfecting said plant cell with an expression cassette in accordance with the first embodiment of the invention, or a vector in accordance with the second embodiment of the invention.

Typically, the plant cell described in the ninth or tenth embodiment of the invention is a meiocyte.

Typically, the method for increasing homologous or homeologous meiotic recombination in a plant cell in accordance with the ninth or tenth embodiments of the invention, further comprises culturing the transformed plant cell under conditions permitting regeneration of a fertile plant.

More typically, the fertile plant regenerated from the transformed or transfected plant cell (meiocyte), is crossed with a plant from a second plant line, to generate a hybrid plant. The hybrid plant line is then permitted to sexually reproduce (preferably by self-fertilisation), and recombination events are identified in the resulting progeny.

Alternatively, the fertile plant regenerated from the transformed or transfected plant cell (meiocyte) is itself a hybrid plant, that is, the plant cell (meiocyte) was obtained from a hybrid plant. In this manner, recombination events may be identified in the regenerated hybrid plant and/or its progeny.

Typically, the method in accordance with any one of the seventh through to tenth embodiments of the invention results in an increase in genetic variation in the plant line wherein homologous or homeologous recombination events have occurred.

Typically, the increase in genetic variation resulting from the homologous or homeologous recombination may be evidenced by new genetic linkage of a desired characteristic trait or gene contributing to a desired characteristic trait.

According to an eleventh embodiment of the invention, there is provided a method for obtaining a plant having a desired characteristic, wherein said method comprises:

a) transforming or transfecting a plant cell or tissue with a polynucleotide encoding a recombinational DNA repair polypeptide, or a fragment thereof, wherein said polynucleotide is capable of stimulating plant meiotic recombination when expressed into RNA and/or said polypeptide, or said polynucleotide is capable of stimulating plant meiotic recombination when introduced into said plant cell or tissue as an RNA::DNA chimeric molecule;

b) culturing said transformed or transfected plant cell or tissue under conditions allowing the regeneration of a plant,

c) permitting said regenerated plant to self-fertilise to produce a first parent line;

d) obtaining a hybrid between a plant of the first parent line and a second parent line, or cells thereof;

e) expressing said polynucleotide in said hybrid plant;

f) permitting said hybrid plant to self-fertilise and produce offspring plants; and

g) screening said offspring plants for plants having said desired characteristic.

According to a twelfth embodiment of the invention, there is provided a method for obtaining a plant having a desired characteristic, wherein said method comprises:

a) transforming or transfecting a plant cell or tissue with an expression cassette in accordance with the first embodiment of the invention, or the vector in accordance with the second embodiment of the invention;

b) culturing said transformed or transfected plant cell or tissue under conditions allowing the regeneration of a plant,

c) permitting said regenerated plant to self-fertilise to produce a first parent line;

d) obtaining a hybrid between a plant of the first parent line and a second parent line, or cells thereof;

e) expressing said polynucleotide in said hybrid plant;

f) permitting said hybrid plant to self-fertilise and produce offspring plants; and

g) screening said offspring plants for plants having said desired characteristic.

The following refers to any one of the seventh through to twelfth embodiments of the invention.

Typically, the polynucleotide capable of stimulating meiotic recombination in plants encodes a recombinational DNA repair polypeptide, or a fragment thereof, selected from the group consisting of: SPO11 (protein ID AAA65532.1), MER1 (NP_{—}014189, MER2 (protein ID AAA34772.1), MRE2 (protein ID BAA02016.1), ME14 (protein ID NP_{—}010963.1), REC102 (protein ID AAA34964.1), REC104 (protein ID AAB26085.1), REC114 (protein ID NP_{—}013852.1), MRE11 (protein ID BAA02017.1), XRS2 (protein ID AAA35220.1), RAD18 (SMC) (protein ID AAA34932.1), RAD50 (protein ID CAA32919.1), RAD51 (protein ID BAA00913.1, AAB37762.1, AAD32030.1, AAD32029.1, AAC23700 or AAF69145.1), RAD52 (protein ID AAA50352.1), RAD54 (protein ID AAA34949.1), RDH54/TID1 (protein ID NP_{—}009629), RAD55-57 (protein ID AAA19688.1, AAA34950.1), DMC1 (protein ID NP_{—}011106.1), and Arabidopsis protein XRS9, or functional fragments or analogues thereof (wherein the protein ID provides a cross-reference to GenBank for the corresponding nucleic acid sequence encoding the relevant polypeptide).

Typically, the polynucleotide capable of stimulating meiotic recombination in plants encoding a recombinational DNA repair polypeptide, or a fragment thereof, is expressed in meiocytes of said plant. Preferably, the polynucleotide is expressed under the control of a promoter of weak to medium strength.

Typically, the polynucleotide capable of stimulating plant meiotic recombination is expressed in the cells of regenerated plant which are capable of undergoing meiosis.

Typically, the plant cell or tissue is selected from any one of the following tissues: leaf, root, seed, stem or flower tissues.

According to a thirteenth embodiment of the invention, there is provided a plant produced in accordance with the method of any one of the seventh through to twelfth embodiments of the invention.

According to a fourteenth embodiment of the invention, there is provided seed from the plant defined in accordance with the thirteenth embodiment of the invention.

According to a fifteenth embodiment of the invention, there is provided the use of a plant as defined in accordance with any one of the fourth, fifth or thirteenth embodiments of the invention, for plant breeding.

### Definitions

The term “nucleic acid” encompasses deoxyribonucleotide (DNA) and/or ribonucleotide (RNA) nucleic acid, either in the single or double-stranded form, and includes within its scope all known analogues of natural nucleotides.

The term “polynucleotide” encompasses deoxyribopolynucleotide and/or ribopolynucleotide, either in the single or double-stranded form, and includes within its scope all known analogues of natural nucleotides. Also, it includes within its scope the relevant sequence as specified, together with the sequence complementary thereto.

As used herein the term “polypeptide” means a polymer made up of amino acids inked together by peptide bonds.

The term “isolated” means that the material in question has been removed from its host, and associated impurities reduced or eliminated. Essentially, it means an object species is the predominant species present (ie., on a molar basis it is more abundant than any other individual species in the composition), and preferably a substantially purified fraction is a composition wherein the object species comprises at least about 30 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into plants. Generally gene transfer involves the process of nucleic acid contact with a target cell by non-specific or receptor mediated interactions, uptake of nucleic acid into the cell through the membrane or by endocytosis, and release of nucleic acid into the cytoplasm from the plasma membrane or endosome. Expression may require, in addition, movement of the nucleic acid into the nucleus of the cell, integration into the host cell's genome, and binding to appropriate nuclear factors for transcription.

The term “expression cassette” refers to a nucleic acid construct comprising a number of nucleic acid elements (promoters, enhancers, the nucleic acid to be transcribed, etc) which permit the transcription of the particular nucleic acid in a host cell. The expression construct can be incorporated into a vector, host chromosome etc.

The term “promoter” refers to nucleic acid sequences that influence and/or promote initiation of transcription.

The term “operably linked” refers to the situation wherein for example, a nucleic acid is “operably linked” when it is placed into a functional relationship with another nucleic acid sequence. For example, a promoter operably linked to a heterologous DNA, which encodes a protein, promotes the production of functional mRNA corresponding to the heterologous DNA.

The term “meiotically active promoter” refers to a promoter which is generally active during prophase of meiosis I, and more specifically active during zygotene and pachytene.

“Conservative amino acid substitutions” refer to the interchangeability of residues having similar side chains. For example, a group of amino acids having aliphatic side is chains is glycine, alanine, valine, leucine, and isoleucine; a group of amino acids having aliphatic-hydroxyl side chains is serine and threonine; a group of amino acids having amide-containing side chains is asparagine and glutamine; a group of amimo acids having aromatic side chains is phenylalanine, tyrosine, and tryptophan; a group of amino acids having basic side chains is lysine, arginine, and histidine; and a group of amino acids having sulfur-containing side chains is cysteine and methionine. Typically, conservative amino acids substitution groups are: valine-leucine-isoleucine, phenylalanine-tyrosine, lysine-arginine, alanine-valine, and asparagine-glutamine.

The tern “transformation” means the alteration of a plant or plant cell genotype by the introduction of exogenous nucleic acid. Typically, the exogenous nucleic acid is stably integrated and expressed in the plant genome.

The term “transfection” refers to the introduction of exogenous nucleic acid into a plant cell, wherein the nucleic acid is either stably integrated into the genome of the plant, or not stably integrated, but merely transiently expressed in the plant.

The term “regeneration” refers to growing a whole plant from a plant cell, a group of plant cells or a plant part.

The term “analogue” as used herein with reference to a nucleic acid sequence means a sequence which is a derivative of the nucleic acid sequences of the invention, which derivative comprises addition, deletion, substitution of one or more bases and wherein the encoded polypeptide retains substantially the same function as the polypeptide encoded by the nucleic acid sequences of defined above. Similarly, the term “analogue” as used herein with reference to a polypeptide means a polypeptide which is a derivative of the polypeptide of the invention, which derivative comprises addition, deletion, substitution of one or more amino acids, such that the polypeptide retains substantially the same function as the polypeptides identified with respect to the first embodiment of the invention.

The term “fragment” of a compound, such as a polypeptide fragment, is a compound having qualitative biological activity in common with for example, the full-length polypeptide.

The term “antisense orientation” refers to a polynucleotide sequence operably linked to a promoter in a manner such that the antisense strand is transcribed. Typically, the antisense strand is complementary to the endogenous transcription product to a degree sufficient such that translation of the endogenous product is substantially inhibited.

In the context of this specification, the term “comprising” means “including principally, but not necessarily solely”. Furthermore, variations of the word “comprising”, such as “comprise” and “comprises”, have correspondingly varied meanings.

### BRIEF DESCRIPTION OF THE DRAWINGS

FIG. 1A: shows the alignment of RAD51 protein sequences from Arabidopsis thaliana (ATRAD51), Zea mais (ZMRAD51A, zmRAD51B) and Lycopersicon esculentum (LERAD51). The alignment was made using the GCG “Pileup” program. Black areas represent identical amino acid residues and white areas indicate non-conservative amino acid changes.

FIG. 1B: illustrates the alignment of RAD51 proteins of Arabidopsis and Homo sapiens using the GCG “Pileup” program. Black and white areas indicate identical and non-identical amino acid positions, respectively. Filled and hatched bars underlining the human sequence indicate the two functional domains in RAD51 protein as described for human RAD51 by Shinohara et al., 1993 (Nature Genet. 4, 239-243) and Aihara et al., 1999 (J. Mol. Biol. 290, 495-504). The N-terminal domain (aa 1 to aa 114) of the human RAD51 is implied in DNA binding, protein-protein interaction, RAD51 regulation, whereas the C-terminal domain (aa 115 to aa 343) shows homology to the central domain of RecA protein and contains ATP binding sites. Since both human and Arabidopsis RAD51 protein show extensive homologies over the entire protein length, a two-domain structure may be deduced for the plant protein.

### SEQUENCE LISTING

SEQ ID NO:1 The sequence (ca 3.3 kb) of the long DMC1 promoter of Arabidopsis thaliana (ecotype Landsberg) is shown. Its core sequence is contained within the DMC1 sequence published by Klimyuk and Jones with Genbank accession number U76670. As described in WO 99/19492, the contents of which are incorporated herein by reference, modifications to the published DMC1 sequence comprise a 5′ terminal SalI restriction site, a modified first exon/intron 1, and the addition of a short polylinker sequence for cloning purposes. Modifications from the description in WO 99/19492 comprise the deletion of the NcoI site in the polylinker as described in Example 2 herein.

SEQ ID NO:2 The sequence (ca. 1.8 kb) of the short DMC1 promoter of Arabidopsis thaliana (ecotype Landsberg) is shown. The sequence core is contained within the DMC1 sequence published by Klimyuk and Jones with Genbank accession number U76670. As described in WO 99/19492, modifications to the published sequence comprise the addition of a 5′ terminal SalI restriction site, the deletion of DNA sequences upstream of the EcoRI restriction site within the promoter, and the addition of a short polylinker for cloning purposes.

SEQ ID NO:3 RAD51 protein sequences from human (hsrad51)

SEQ ID NO:4 RAD51 protein sequences from Lycopersicon esculentum (LERAD51)

SEQ ID NO:5 RAD51 protein sequences from Zea mais (ZMRAD51A)

SEQ ID NO:6 RAD51 protein sequences from Zea mais (zmRAD51B)

SEQ ID NO:7 RAD51 protein sequences from Arabidopsis thaliana (ATRAD51)

### BEST MODE OR MODES OF PERFORMING THE INVENTION

In carrying out the present invention, one of the first steps is the preparation of the relevant expression cassette. In terms of the production of the expression cassette of the present invention, techniques are well known to the person skilled in the art, and enabling instructions are provided in technical manuals such as Sambrook et al. (1989); Molecular Cloning, A Laboratory Manual, 2nd edition, Cold Spring Harbour, N.Y. Further, there are a variety of means of ligating nucleic acid fragments, wherein the choice is often determined by the nature of the termini of such DNA fragments. For example, DNA termini may be joined using either T4-DNA ligase or Taq-DNA ligase. Complementary overhanging termini of purified DNA are usually ligated without pretreatment, whereas incompatible termini have to be blunted before ligation either by receding endonuclease action or by proceeding fill-in action.

In constructing the expression cassette, it is also preferable to provide appropriate ribosome binding sites, transcription initiation and termination sequences, translation initiation and termination sequences and polyadenylation sequences, thereby permitting the production of a functional RNA transcript which can be translated into a recombinational DNA repair polypeptide.

Once an expression cassette is constructed, the transformation of plants can be carried out in accordance with the invention by essentially any of the various transformation/transfection and regeneration methods known to those skilled in the art. Examples of these methods are described in Methods and Enzymology, vol. 153, 1987, (Wu and Grossman, eds) Academic Press, the disclosure of which is incorporated herein by reference. For example, methods of plant transformation useful in the present invention include: ballistic and chemical methods, use of viral vectors, electoporation of protoplasts, Agrobacterium tumefaciens or Agrobacterium rhizogenes mediated gene transfer. Examples of a procedure which may be followed to introduce such nucleic acid sequences encoding proteins which act in recombinational DNA repair are outlined in Examples 3, 5 and 8.

More specifically, these examples of methods for transformation of plant cells include the direct microinjection of nucleic acid into a plant cell by use of micropipettes. As far as chemical methods of transformation are concerned, one example by which nucleic acid can be transferred into a plant cell is by polyethylene glycol, and this is outlined in Paszkowski et al. EMBO J. 3: 2717-2722 (1984), the contents of which are incorporated herein by reference.

In terms of electroporation of protoplasts, reference is made to Fromm, et al. Proc. Natl. Acad. Sci. U.S.A. 82: 5824 (1985), the contents of which are incorporated herein by reference.

Transformation may also be carried out via infection with a plant specific virus, e.g., cauliflower mosaic virus, and this is described in Hohn et al. “Molecular Biology of Tumors”, Academic Press, New York (1982), pp. 549-560, the contents of which are incorporated herein by reference. The use of viral vectors relies on the viral vector replicating as an extrachromosomal nucleic acid (DNA or RNA) molecule. In this way, the person skilled in the art can prepare a shuttle-type vector comprising the essential viral sequences critical to replication, and such vectors can also include the expression construct of the present invention as exogenous nucleic acid material, thereby providing a mechanism to integrate the expression construct into plants and plant cells. Examples of such vectors include geminiviruses such as wheat dwarf virus. These can be transformed into the plant cell nucleus, wherein they propagate to high copy number. Such high-copy number increase the chance that a recombination event will occur between the target sequence and the construct, leading to successful construct integration. Alternatively, certain plant viral vectors may be used to overexpress the exogenous nucleic acid material directly in the plant cytoplasm without prior integration into the plant genome. Such viral vectors include potyviruses (T. Dalmay et al., 2000, Plant Cell 12, 369-379; S. M. Angell and D. C. Baulcombe, 1999, Plant Journal 20, 357-362).

A still further method of transformation of plant cells involves the introduction of nucleic acid contained within the matrix or on the surface of small beads or particles way of high velocity ballistic penetration of the plant cell, and this is described in Klein et al. Nature 327: 70-73 (1987), the contents of which are incorporated herein by reference.

Alternatively, a person skilled in the art can make use of transformation sequences of plant specific bacteria such as Agrobacterium tumefaciens, e.g., a Ti plasmid transmitted to a plant upon infection by Agrobacterium tumefaciens, and this technique is described in Horsch et al. Science 233: 496-498 (1984); Fraley et al. Proc. Natl. Acad. Sci. U.S.A. 80: 4803 (1983), the contents of which are incorporated herein by reference.

More specifically, the use of Agrobacterium-mediated gene transfer techniques relies on the ability of Agrobacterium tumefaciens to transfer DNA into plants. Agrobacterium is a plant pathogen, and it transfers the T-region of the Ti plasmid within Agrobacterium, into the host plant genome, via infection at wound sites in the plant. The Agrobacterium-mediated gene transfer/infection results in crown gall disease, and involves the stable integration of T-DNA into the plant genome. Furthermore, the ability to produce crown gall disease can be removed by deletion of tumuorogenic disease from the T-DNA, wherein the T-DNA is engineered in such a way as to maintain the DNA transfer and integration function. Consequently, the expression construct of the present invention to be inserted into the plant of interest is attached to the border sequences of the T-DNA, and is inserted accordingly.

Further, supervirulent strains of Agrobacterium have been engineered to utilise Agrobacterium as a vector for use with both monocotyledonous and dicotyledenous plants. Examples of highly virulent Agrobacterium stains include among others AGL1, and EHA101 (E. E. Hood et al., 1986, J.Bacteriol. 168, 1291-1301; G. R. Lazo et al., 1991, Bio/Technology 9, 963-967).

In the transformation of meiocytes in accordance with the ninth or tenth embodiment there are two alternative outcomes in terms of the fertility of the plant regenerated.

In general, if the plant cell completes meiosis following recombination, the regenerated plant will be haploid and thus infertile. However, fertility may be restored by doubling the chromosome number of said plant using colchicine treatments known in the art as “Double Haploidisation”, as described in B. Barnabas et al., 1999, Plant Cell Reports 18, 858-862; J. Zhoa et al., 1996, Plant Cell Reports 15, 668-671; L. Alemanno and E. Guiderdoni, 1994, Plant Cell Reports 13, 432-436; Y. Wan et al., 1989, Theor. Appl. Genet 77, 889-892; the disclosures of which are incorporated herein by reference.

However, if the plant cell does not complete meiosis, the regenerated plant will be diploid and fertile, and will not require colchicine treatment.

After the vector comprising the expression cassette of the invention is introduced into a plant cell, selection for successful transformation is often carried out prior to and/or during regeneration of a plant. An example of such a selection technique is one based on antibiotic or herbicide resistance and/or resistance genes which may be incorporated into the transformation vector.

Methods for plant regeneration are well known to those skilled in the art. For example, regeneration of cultured protoplasts is described by Evans et al. “Protoplasts Isolation and Culture”, Handbook of Cell Cultures 1: 124-176 (MacMillan Publishing Co., New York (1983); and H. Binding “Regeneration of Plants”, Protoplasts, pp. 21-73 (CRC Press, Bocaraton 1985), the contents of which are incorporated herein by reference. Yet other techniques are described in Examples 3, 5 and 8.

Once transformed, a transgenic plant containing such an introduced expression construct can be bred true to obtain a homozygous line which expresses the recombinational DNA repair gene in meiocytes of male or female reproductive organs, and preferentially in both. This homozygous line then serves as a first parental plant in crosses to a second parent plant, wherein the second parent usually is the pollen donor. This second parent plant is member of a species which is identical or closely related to the species of the first parental plant, so that both parents are sexually compatible. As F1 progenies express the gene encoding the recombinational DNA repair polypeptide as a heterozygous trait, the frequency of meiotic recombination between homologous or homeologous parental chromosomes become elevated at prophase of F1 meiosis. Because fertility is not disabled in F1 plants, F2 seed can be obtained, usually after self-fertilisation.

Recombination events are then identified among F2 progenies with eater probability than normally expected, and this is evidenced in Example 7 herein. In consequence, less parental crosses need to be performed and/or less F2 progenies need to be screened for rare recombination events between two target loci.

A time saving alternative method of producing F1 plants which express the recombinational DNA repair gene at meiosis consists of crossing the transgenic plant, usually as the female parent, directly to the second parent plant. In this method, the transgenic parent plant usually is heterozygous for the expression construct and only a proportion of F1 offspring will thus inherit the transgene. However, these can be identified conveniently for example by making use of a selection marker that is genetically linked to the expression construct, and reference to this is made in Example 8.

According to the present invention, the expression cassette inserted into the plant genome is comprised of a construct, the construct comprising a meiotically active promoter sequence (preferably a meiosis specific plant promoter sequence), operably linked to a polynucleotide encoding a recombinational DNA repair polypeptide, or fragment thereof, wherein said polynucleotide is capable of stimulating recombinational DNA repair when expressed into RNA and/or said polypeptide. The meiotically active promoter sequence confers expression of the polynucleotide during meiotic prophase I, specifically during zygotene and pachytene when chromosome synapsis and meiotic recombination occur.

As described above, and in accordance with the first embodiment of the invention, the polynucleotide capable of stimulating plant meiotic recombination by encoding a recombinational DNA repair polypeptide, or fragment thereof, may be present in either sense or anti-sense orientation, with respect to the meiotically active promoter. Coding in the anti-sense orientation permits the downregulation of expression of any endogenous recombinational DNA repair polypeptide.

Conversely, coding in the sense orientation may potentially lead to either of two outcomes. Firstly, translation and thus expression of the recombinational DNA repair polypeptide. Secondly, down-regulation of both exogenous and endogenous recombinational DNA repair polypeptide expression through co-suppression or post-transcriptional gene silencing, as described below. Further, co-suppression is described in the following references: T. Elmayan et al., 1998, Plant Cell 10, 1747-1758; Q. Que and R. A. Jorgensen, 1998, Dev. Genet. 22, 100-109; P. M. Waterhouse, 1998, PNAS 10, 13959-13964, the contents of which are incorporated herein by reference.

Both outcomes may stimulate meiotic recombination depending on the natural biological function of the polypeptide in question. Where polypeptides naturally support or execute recombination, it can be of advantage to add or overexpress them at meiosis. However, in cases where polypeptides normally are antagonistic to interhomologue recombination at meiosis, their expression may be down-regulated during meiotic prophase to stimulate recombination by either antisense or co-suppression technology. Further to this, where gene inactivation is the desired outcome, ribozyme technology (J. Haselhof and W. L. Gerlach, 1988, Nature 334, 585-591), virus induced gene silencing (VIGS) technology (D. C. Baulcombe, 1999, Current Opinion in Plant Biology 2, 109-113), post transcriptional gene silencing (PTGS) technology (A. Depicker and M. Van Montagu, 1997, Current Opinion in Cell Biology 9, 372-382), double-stranded RNA technology (C. F. Chuang and E. M. Meyerowitz, 2000, PNAS 97, 4985-4990), antibody technology (A. Hiatt et al., 1989, Nature 342, 76-78), or chimeric RNA::DNA oligonucleotides (r. Zhu et al., 1999, PNAS 96, 8768-8773; K. Yoon et al., 1996, PNAS 93, 2071-2076; A. Cole-Strauss, 1996, Science 273, 1386-1389), may also be employed as gene inactivation tools, or any other technique known to the person skilled in the art, wherein the contents of each of the above references are incorporated herein by reference.

Further to this, the RNA capable of stimulating plant meiotic recombination by encoding a recombinational DNA repair polypeptide, or fragment thereof, is not necessarily limited to mRNA. For instance, when vectors are used that are derived from RNA viruses, the polynucleotide encoding the recombinational DNA repair polypeptide does not need to be expressed as mRNA in order to downregulate the expression of the endogenous gene. Rather, it is enough to express the RNA as part of the viral RNA, in a manner as described in the references outlined above.

Preferably, the expression construct of the present invention comprising the polynucleotide sequence, or part thereof, capable of stimulating meiotic recombination, is derived from plants and is shown to function therein during meiotic recombination. However, in cases where a polynucleotide sequence of non-plant source can also function in plant meiotic recombination, it may be used in plants to elevate the level of recombination for plant breeding purposes.

Further, with respect to the polynucleotide capable of stimulating plant meiotic recombination being present as an RNA::DNA chimeric molecule, the molecule has a mode of action on meiotic recombination as follows:

The RNA::DNA chimeric molecule provides a vehicle for introducing point mutation(s) into target genes in a sequence dependent manner via gene conversion. This technique of in vivo mutagenesis is generally known as “Kimeragen” technology, and the following references describing this technology are incorporated herein by reference (K. Yoon et al., 1996, PNAS 93, 2071-2076; A. Cole-Strauss et al., 1996, Science 273, 1386-1389).

“Kimeragen” technology can be applied to plants (T. Zhu et al., 1999, PNAS 96, 8768-8773), whereby the RNA::DNA chimeric molecule is introduced into plant cells by particle bombardment or other methods suitable for introduction of nucleic acids into plant cells. Upon introduction of the RNA::DNA chimeric molecules, the target gene sequence is modified in a proportion of cells. Consequently, progenies of cells treated in this manner can be selected for (depending on the introduced mutation) or be identified by PCR and DNA sequencing. Eventually, plants containing the mutation can be regenerated from these cells using standard techniques of plant regeneration. Mutations thus introduced into plants will be stably inherited in a mendelian fashion. If the mutation was designed to inactivate an endogenous gene function which normally hinders, reduces or controls meiotic recombination, then the regenerated mutant plant and its progenies will show an elevated level of meiotic recombination and will thus be useful in conjunction with any one of the seventh to twelfth embodiments of the invention. In essence, Kimeragen technology can be used as an alternative to other techniques which aim at down regulating or inactivating the expression of an endogenous target gene in a plant.

Whilst it may be necessary for down-regulation of gene expression to isolate the relevant polynucleotide sequence from the target plant species of interest, this is usually not required when such polynucleotide sequences from other plant sources are available and are to be translated into protein. Due to high sequence conservation between homologous DNA repair proteins of different species, polynucleotide sequences involved in recombinational DNA repair can be exchanged between plant species without noticeable loss of protein function, provided that their resulting protein sequence does not typically diverge by more than about 45%, more typically, about 40%, even more typically, about 35%, still more typically, about 30%, and yet still more typically, about 25%.

For example, the RAD51 protein of Arabidopsis is 86% identical to maize RAD51, and as outlined in Example 1, both proteins share an overall similarity of 93%. Therefore, RAD51 protein of Arabidopsis will thus be functional not only in the species of the Brassicaceae family, but also in the species of the Gramineae family, including Zea mais.

A preferred polynucleotide sequence involved in meiotic recombination for use in the present invention is one encoding the complete plant RAD51 protein, and as outlined in Example 5, is inserted in sense orientation with respect to the promoter. Whilst it may be preferable that the RAD51 sequence be derived from Arabidopsis thaliana, RAD51 sequences of other plant species will be equally useful in sense orientation, as will be RAD51 sequences of non-plant sources.

Also useful in the present invention will be other polynucleotides capable of stimulating plant meiotic recombination, such as those encoding plant homologues of other yeast DNA recombination repair functions. Examples of such polynucleotides include those encoding plant homologues of: SPO11, MER1, MER2, MRE2, MEI4, REC102, REC104, REC114, RAD18 (SMC), RAD50, Rad52, RAD54, RDH54/TID1, RAD55-57, MRE11, XRS2, DMC1, or of Arabidopsis XRS9.

Alternatively, and as described above, polynucleotides capable of stimulating plant meiotic recombination for use in the present invention may contain, in antisense orientation, the open reading frame, or part thereof, of Arabidopsis XRS4, or of other plant homologues of XRS4, or for that matter any plant protein which normally hinders meiotic recombination. Further still, another expression construct of the present invention may comprise polynucleotide sequences which encode a ribozyme or an antibody directed against XRS4 mRNA or protein respectively, or against any other plant mRNA or protein which normally hinders meiotic recombination in plants. Further to this, where gene inactivation is the desired outcome, ribozyme technology, virus induced gene silencing (VIGS) technology, co-suppression, post transcriptional gene silencing (PTGS) technology, double-stranded RNA technology, antibody technology, or chimeric RNA::DNA oligonucleotides may also be employed as gene inactivation tools, or any other technique known to the person skilled in the art, wherein the reference for each technology is provided above.

According to the present invention, the polynucleotide sequence involved in recombinational DNA repair within the expression construct is placed under operative control of a meiotically active, preferably, meiosis specific promoter which is active during zygotene and pachytene of meiosis I. When the expression construct comprises recombinational DNA repair polynucleotide sequences of plant origin, and these sequences are naturally expressed at meiosis during this time interval, the promoters of these polynucleotides can be used.

However, it should be noted that meiotically active promoters of a proportion of DNA repair genes may also be active in somatic cells, and often are inducible by DNA damage. Consequently, these promoters could be difficult to control under growth conditions in the field, and although still useful for the purpose of the present invention, they are less preferable than meiosis specific promoters.

Essentially, any meiotically active, preferably, plant meiotically active promoter which confers gene expression at zygotene and pachytene of meiosis I may be useful in the present invention. However, the most preferable plant promoter for use in the expression construct of the present invention is one specific to meiosis, and one which is not or not predominantly active in the somatic tissues of a plant. The advantage of such a promoter is in avoiding the potential cytotoxic effects of the expression of the polynucleotide sequences involved in recombinational DNA repair on plant growth and development.

As exemplified in Example 3, the preferred plant promoter is of weak to medium strength, thereby not exerting any potential cytotoxic effects on meiocyte development, since such activity could sterilise the plant. As described in Example 4, results obtained in living cells of Escherichia coli may indicate an undesirable effect of strong RAD51 expression.

Therefore, one of the most preferred plant promoters of the present invention is the short or long promoter versions of the DMC1 gene of Arabidopsis thaliana (L. er), and reference is made to Example 2 for a description of the production of these promoter types. Of course, similar promoter versions can be derived by polymerase chain reaction (PCR) techniques known to the person skilled in the art from DMC1 homologues of other ecotypes or of other plant species, preferably the species of interest.

The invention will now be described in greater detail by reference to specific Examples, which should not be construed as in any way limiting the scope of the invention.

### EXAMPLES

### Example 1

Sequence Comparison Between RAD51 Proteins of Arabidopsis, Maize and Tomato.

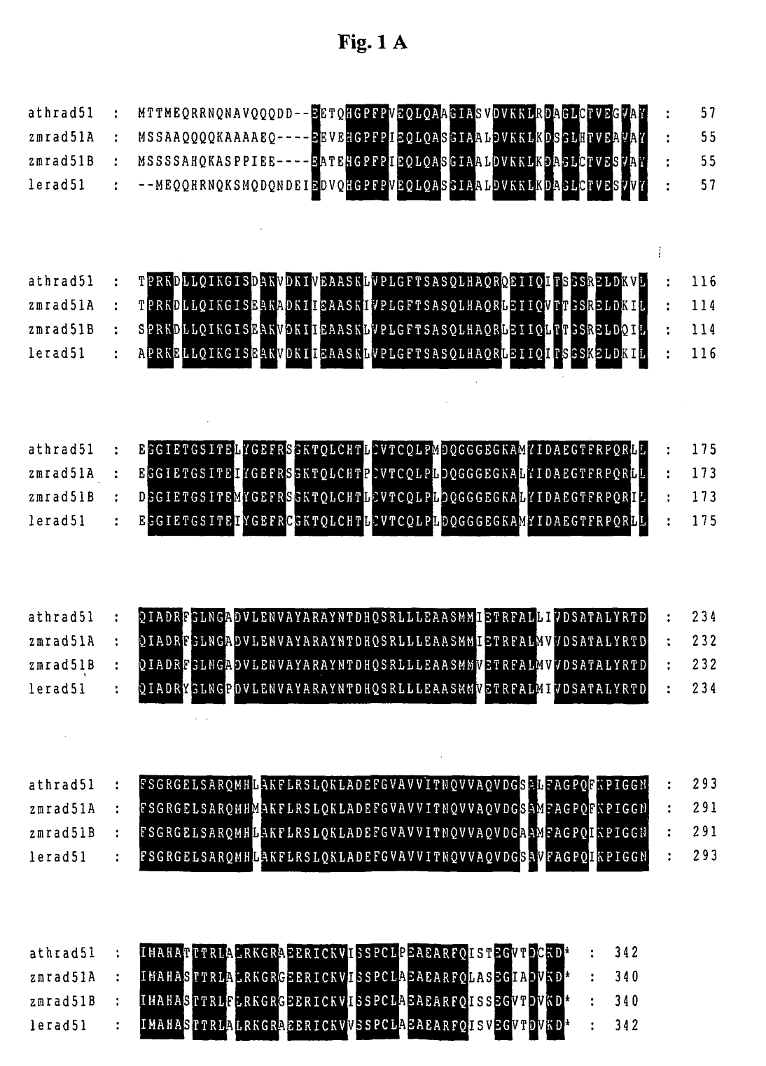

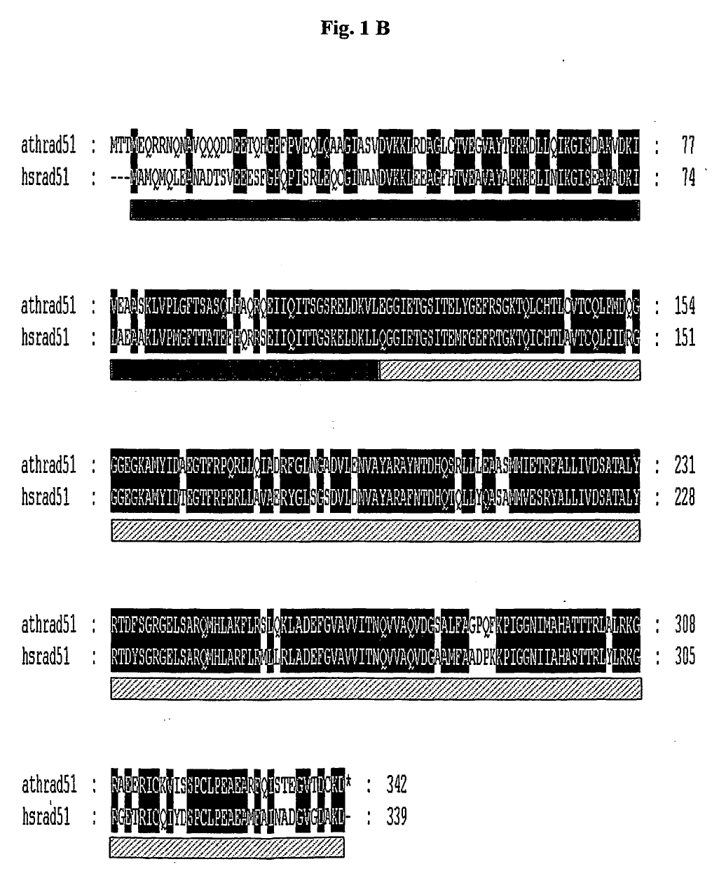

The GCG-Pileup program was used to make a sequence alignment (FIG. 1A of different RAD51 proteins found in Arabidopsis thaliana (AtRAD51 ecotpe “columbia”, Genbank protein ID: CAA04529; and AtRAD51 ecotype , “landsberg erecta”, Genbank protein ID: AAB37762). Zea mais (ZMRAD51A, Genbank protein ID: AAD32029; ZmRAD51b, GenBank protein ID: AAD32030), and of Lycopersicon esculentum (LeRAD51, Genbank protein ID: AAC23700).

Using the GCG-GAP program protein similarity and identity values were calculated as follows with resect to AtRAD51 protein (Genbank protein ID: CAA04529). For completeness similarity and identity values were also calculated for RAD51 of Saccharomyces cerevisiae (Genbank protein ID CAA45563.1):

ZmRAD51A: 92.7% similarity, 86.2% identity

ZmRAD51B: 93.3% similarity, 86.2% identity

LeRAD51: 96.2% similarity, 88.9% identity.

yRAD51: 77.3% similarity, 62.7% identity.

As shown in FIG. 1A, significant sequence differences among the listed plant RAD51 proteins can be seen only at the extreme NH₂ terminus. As such differences also occur between the two proteins of maize, they are not regarded as relevant to protein function. The remaining RAD51 sequence is highly conserved among the listed plant proteins which indicates that RAD51 function is interchangeable between plant species without significant loss of protein function.

FIG. 1B shows a sequence alignment of Arabidopsis RAD51 (Genbank protein ID: CAA04529) to human RAD51 (Genbank protein ID: AAF69145), which is well characterised as having two functional protein domains (amino acid positions 1-114 and amino acid positions 115-339). Its N-terminal domain is implicated in DNA binding, protein-protein interaction and RAD51 regulation, whereas its C-terminal domain is homologous to the central part of the bacterial REC A protein and contains several ATP binding sites (Shinohara et al., 1993, Nature Genet. 4, 239-243; H. Aiham et al., 1999, J. Mol. Biol. 290, 495-504). Given the strong similarity of plant and human RAD51 protein, a similar domain structure may be deduced for Arabidopsis RAD51.

### Example 2

Construction of Arabidopsis thaliana Meiocyte Specific Expression Cassettes

1. Short DMC1 Promoter Constructs

As described previously in Australian Patent Application No. 11573/99 (WO 99/19492), the contents of which are incorporated herein by reference, the short DMC1 promoter version (1.8 kb) was obtained by PCR from genomic DNA of Arabidopsis thaliana (ecotype Landsberg), and cloned into p2030 to yield the high copy number plasmid p2031. The latter plasmid is used herein as a starting material for construction of RAD51 and GUS expression cassettes as follows:

(i) GUS Expression Cassette:

The 1.8 kb long promoter fragment was recovered from p2031 after digestion with SalI and SmaI, and was cloned in between the restriction sites of SalI and SmaI of pBI101.3 (R. A. Jefferson, 1987, Plant Mol. Bio. Rep. 5, 387-405) to give plasmid p2042. The latter plasmid was used for plant transformation.

(ii) RAD5 Expression Cassette:

The full-length cDNA of Arabidopsis thaliana (ecotype Columbia) was isolated and cloned into the SmaI site pUC18 as described by M. P. Doutriaux et al. (1998, Mol. Gen. Genet 257, 283-291) to give plasmid pRAD51. The cDNA was recovered from pRAD51 after plasmid digestion with KpnI (at 5′end) and BamHI (at 3′ end) and was gel purified. The isolated fragment was then cloned in two subsequent ligation steps into plasmid p2031 which had been opened with KpnI and had been treated with Shrimp Alkaline Phosphatase to avoid re-ligation of the vector. In the first ligation step the compatible KpnI ends of insert and vector were joined. This ligation was followed by a fill-in reaction of the non-ligated ends using Klenow fragment of E.coli DNA polymerase I to produce a blunt end in the insert and in the vector, respectively. A subsequent ligation step then joined these blunted ends. The ligation products were introduced into E.coli and clones containing the RAD51 cDNA in either sense or antisense orientation with respect to the DMC1 promoter were identified using a diagnostic HindIII digestion.

Cloning in sense orientation yielded one 1.9 kb and one 4.3 kb DNA fragment whereas cloning in antisense orientation yielded one 2.7 kb and one 3.5 kb DNA fragment. The plasmid containing the cDNA in antisense orientation was named p2035, while the plasmid containing the cDNA in sense orientation was designated p2034. Eventually, the (short) P_{DMC1}::Rad51::nosT expression cassette of p2034 was transferred as an EcoRI fragment (3 kb) into the EcoRI restriction site of the binary vector pNos-Hyg-SCV. The final product was designated as plasmid p3243 and contained the (short) P_{DMC1}::Rad51::nosT gene in the same direction of transcription as the upstream located hygromycin resistance gene.

2. Long DMC1 Promoter Constructs

The isolation of the two components of a long DMC1 promoter version (3.3 kb) by PCR from Arabidopsis thaliana (ecotype Landsberg), and how to combine and clone them into a binary transformation vector is also described. These components comprise a promoter sequence (3.1 kb) as a SalI/XbaI DNA fragment and the modified first exon/intron of the DMC1 gene (ca 0.2 kb) as a XbaI/SmaI fragment. The modification consisted in replacement of the first two amino acid codons in exon 1 (. . . ATG ATG . . . ) with an XbaI restriction site, and of a short polylinker downstream of the 3′ splice site of intron 1. Both components are used here as a starting material for construction of RAD51 and GUS expression cassettes as follows:

(i) General Expression Cassette:

The PCR fragment comprising the promoter without the modified first exon/intron 1 (3.1 kb) was digested with SalI/XbaI and was cloned between the restriction sites for SalI and XbaI of pBS(SK+) to yield plasmid p2060. The PCR fragment comprising the modified first exon/intron (ca. 0.2 kb) was digested with XbaI and SmaI and was cloned separately into pBS(SK+) predigested with XbaI and SmaI, to give plasmid p2061. For the purpose of combining the two components, the promoter fragment (recovered from p2060 as a SalI/XbaI fragment) and the modified exon/intron (isolated from p2061 as a XbaI/KpnI fragment) were added in a single step ligation to vector pBS(SK+) which had been opened for this purpose with restriction enzymes SalI and KpnI. The cloning yielded plasmid p4904, which contained an NcoI restriction site in the polylinker of the future expression cassette. This site was deleted from p4904 by treatment with Mung Bean nuclease which removed the overhanging bases from the NcoI ends. Re-ligation of the plasmid generated plasmid p4907. The region comprising the combined long promoter, exon/intron and the polylinker was then excised from p4907 by digestion with SalI and SmaI and was cloned immediately upstream of the NOS terminator into p3264 which had been pre-digested with SalI and SmaI. This cloning yielded a complete expression cassette for general cloning purposes in a pBIN19 vector backbone.

(ii) RAD51 Expression Cassette:

pRAD51 was digested with Acc65I and SalI to isolate the RAD51 encoding cDNA. The ends were filled-in with Klenow fragment of E.coli DNA polymerase I to allow cloning into the blunted (described above) NcoI site of p4904. The new plasmid was called p4926. A SalI/EcoRV DNA fragment comprising the long DMC1 promoter, exon/intron and RAD51 cDNA in sense orientation, was then cloned into p3264 which had been predigested with SalI and SmaI to complete the RAD51 expression cassette in a pBIN19 vector backbone. This step yielded plasmid p4928.

(iii) GUS Expression Cassette Minus Exon/Intron:

The 3.1 kb SalI/XbaI fragment of p2060 comprising the long DMC1 promoter version minus exon/intron was cloned between the restriction sites of SalI and XbaI of pBI101.3 to generate plasmid p3276; it contains between the T-DNA borders a complete GUS expression cassette in a pBIN19 vector backbone.

(iv) GUS Expression Cassette Including Exon/Intron:

The 3.3 kb SalI/BglII fragment of p4907 comprising the long DMC1 promoter version including exon/intron was cloned between the restriction sites of SalI and BaHI of pBI10.3 to generate plasmid p3277; it contains between the T-DNA borders another complete GUS expression cassette in a pBIN19 vector backbone.

### Example 3

In Plant Evaluation of Melocyte Specific Expression Cassettes Using Transcriptional Fusions to GUS.

The plasmids p3276 and p3277, containing each the long DMC1 promoter version once without and once with exon/intron, respectively, were introduced by electoporation intro Agrobacterium tumefaciens strain AGL1. Electroporation of Agrobacterium was performed with a BIORAD GENE PULSER as for E.coli transformation (W. J. Dower, Electroporation of Bacteria, In “Genetic Engineering” vol. 12, Plenum Press, New York 1990, J. K Setlow eds.) except that car 10 ng of plasmid DNA was used per electroporation. After 2 days at 28° C. transformed Agrobacterium colonies were visible on kanamycin containing (50 mg/L) LB medium plates. Transformed Agrobacterium clones were grown in LB liquid medium and used for in planta transformation of Arabidopsis thaliana (ecotype C24) using the “Simplified Arabidopsis Transformation Protocol” of A. Bent and S. Clough as published (In: Plant Molecular Biology Manual, 2nd ed., 1998; S. B. Gelvin and R. A. Schilperoort, eds., Kluwer Academic Pub., Dordrecht, NL). Thus treated plants (T0 plants) were allowed to self-fertilise to give rise to T1 seeds, which after surface sterilisation were plated onto MS germination medium (reference D. Valvekens et al., 1988, PNAS 85, 5536-5540) contaning kanamycin (50 mg/L) for selection of transformed plants. Kanamycin resistant plantlets were transferred to soil and grown to maturity. Flower tissue was harvested from early stages of flower development through to seed set and was processed for histochemical staining of GUS activity as is described by R. A. Jefferson et al. (1987, EMBO J. 6, 3901-3907).

The latter analysis revealed that GUS activity was very low in plants expressing GUS from the short DMC1 promoter of p2042 (11 primary transformants). In contrast, both long promoter versions (p3276 and p3277) gave strong X-Gluc staining in both meiotic anthers and ovules in agreement with data previously described with a DMC1 promoter version of Klimyuk and Jones (V. I. Klimyuk and J. D. G. Jones, 1997, Plant J. 11, 1-14). There was no detectable difference between the intron-plus (25 primary transformants) and the intron-minus promoter version (16 primary transformants). However, transformation with either long promoter version also yielded plants with unexpected expression pattern. For example a small number of plants showed X-Gluc staining only in meiotic anothers, and a majority of plants showed GUS activity not only in meiotic anther and meiotic ovule, but to various degree also in other floral tissues/organs, such as tips of sepals and petals, carpel walls, stigma, transmitting tract, and even wounding sites.

According to these observations both long promoter versions give satisfactory results with regard to meiotic gene expression, even though the promoters are not entirely specific to meiosis. Furthermore, availability of short and long promoter versions allow the experimentalist to choose between meiotic promoters of various strength. The weak short promoter version may be particularly unsell where strong transgene expression is suspected to be detrimental to the host cell. The long promoter versions may substitute for the short version where the latter is too weak to yield sufficient transgene expression. Finally, the intron containing long promoter may be especially useful to drive transgene expression in monocotyledonous plants such as maize where the addition of an intron to the promoter can increase gene expression (D. McElroy et al., 1991, Mol. Gen. Genet. 231, 150-160).

### Example 4

Cytotoxicity of RAD51 Overexpression in Escherichia coil

As described above (Example 2.1.(ii)) the RAD51 cDNA was subcloned in two ligation steps as a KpnI/BamHI fragment into the KpnI site of the P_{DMC1}::nosT expression cassette on p2031. Since the first ligation step involved the joining of KpnI ends the cDNA was expected to insert at equal ratio in both orientations with respect to the DMC1 promoter. However, later restriction analysis with HindIII of plasmid DNA from 14 randomly chosen E.coli transformants (strain:XLBlue1) revealed a bias of insert orientation. More specifically, out of 14 clones, four clones were discaded as cloning artefacts, nine clones contained inserts in sense orientation and only one clone contained the cDNA in antisense orientation. This distribution compares unfavourably with the expected 1:1 ratio of sense to antisense orientation (Chi²=6.4, P=0.01). A possible explanation for this insertion bias may arise from alignment of the inset with respect to the bacterial LacZ promoter in p2031, which reads in opposite direction to P_{DMC1}. Such comparison reveals an orientation bias 9:1 in favour of antisense orientation with respect to the P_{lacz}. These data suggest that plant RAD51 protein may be cytotoxic to E. coli and possibly also to other living cells when overexpressed from strong promoters.

This interpretation is supported by another line of evidence, which is described below. AtRAD51 was cloned in frame with the ATG start-codon of pSE380 (Invitrogen) and of pTYB11 (New England Biolabs), respectively, both of which are bacterial expression vectors. These clonings were done as follows. The cloned AtRAD51 in pRAD51 (M. P. Doutniaux et al., 1998, Mol. Gen. Genet. 257, 283-291) was modified by PCR using the Pfu thermostable polymerase from “Stratagene”, the forward primer RADNCO (5′-CATGCCATGGGAATGACGACGATGGAGCAGCGTAG-3′) and the reverse primer RADDOECO: 5′-ATGAATTCGGATCAATCCTTGCAATCTGTTACACC-3′. The resulting PCR product was cloned at first into the SmaI site of pBS(KS), then re-isolated from pBS(KS) as an NcoI-EcoRI fragment and cloned between the NcoI and EcoRI sites of pSE380. Another PCR induced modification was made with Pfu polymerase on the same pRAD51 template but using the forward primer RADSAP: 5′-GGTGGTTGCTCTTCCAACATGACGACGATGGAGCAGCGTAG-3′ in conjunction with the reverse primer RADDOECO (above). The DNA product resulting from this latter PCR reaction was also cloned initially into the SmaI site of pBS(KS), but was then re-isolated from PBS(KS) as a 1 kb fragment after partial digestion with SapI and EcoRI. The fragment was subsequently cloned between the SapI and EcoRI sites of pTYB11.

The cloning in pSE380 allowed IPTG inducible expression of active RAD51 protein, whereas the cloning in pTYB11 was designed to yield IPTG inducible expression of inactive RAD51::INTEIN fusion protein. These recombinant plasmids were transformed by electroporation into E.coli strains, which were proficient or deficient respectively for the bacterial recombination function REC A. Control transformations were carried out with the empty vector pSE380. For plasmid maintenance, transformants were selected on ampicillin containing LB medium in the absence of IPTG. A single colony was randomly chosen from each transformation experiment and was grown overnight to comparable optical densities in liquid LB medium containing ampicillin but lacking IPTG. Appropriate dilutions (in sterile LB medium) of each overnight culture were then plated onto LB plates containing or lacking IPTG, respectively. After overnight incubation at 37° C. the number of colonies growing on plates with or without IPTG was compared.

Table 1 describes the cytotoxicity of plant RAD51 expression in Escherichia coli based on colony counts expressed as “colony forming units per millilitre” (cfu/ml). As shown, IPTG induction had no effect on growth of transformants expressing either RAD51::INTEIN fusion protein or no RAD51 protein at all (control). In contrast, the colony count decreased several 1000-fold in transformants expressing AtRAD51 upon IPTG induction.

These results were independent of the genetic background of the host strain Furthermore, the cytotoxicity to E.coli of plant RAD51 thus demonstrated might even be an underestimation as indicated by colony replication from IPTG containing plates onto fresh IPTG plates (+IPTG #2): colony regrowth was found only with 5 out of 181 colonies and with 4 out of 155 colonies, respectively. Additionally, only 1 out of each set of surviving colonies produced ethidium bromide detectible plasmid DNA.

### Example 5

Plant Transformation with (Short) P_{DMC1}::Rad51::nosT

Plasmid p3243 containing the (short) P_{DMC1}::Rad51:nosT expression cassette between its T-DNA borders was introduced into Agrobacterium tumefaciens strain AGL1 using the electroporation protocol described in Example 3. Transgenic Agrobacteria were selected on LB medium containing Kanamycin (15 mg/L). One Agrobacterium clone containing plasmid p3243 was chosen at random to transform Arabidopsis thaliana (ecotype C24) using the root transformation protocol of Valvekens (D. Valvekens et al., 1988, PNAS 85, 5536-5540). Transgenic plantlets were regenerated in vitro on appropriate tissue culture media as described by Valvekens (above) but containing Hygromycin (30 mg/L) instead of Kanamycin. Plantlets were transferred to soil once their root systems were well developed. Five independent transformation events (A to E) were obtained and grown to maturity. All plants were fertile and, after self-fertilisation, produced seed (T1 progeny).

### Example 6

Generation of F2 Screening Populations for Recombination Analysis

Two transgenic lines (A and E) of Example 5 were selected for later recombination experiments. The pattern of T-DNA integration within lines A and E was determined by Southern Blot analysis of hygromycin resistant T1 and T3 progenies. This analysis indicated a single T-DNA insertion in line A. Analysis of hygromycin resistance segregation then led to identification of a homozygous T1 individual A7, a T2 progeny of which was crossed as the female to CS10, which is an Arabidopsis thaliana line of ecotype Landsberg (Nottinngham Arabidopsis Stock Centre, NASC). For the purpose of crossing, the male parent was grown at high humidity in order to overcome the reduced male fertility phenotype associated with the cer mutation in CS10. The crossing was done manually by removing with forceps dehiscent anthers of CS10 plants and brushing them over the pistil of an emasculated flower from the female parent plant at high air humidity. The crossing resulted in F1 offspring A7-2 to A7-5 which were allowed to self-fertilise and produce F2 progeny. The corresponding F2 screening populations segregated the hygromycin resistance gene at 3:1 ratios in three out of four populations analysed (A7-2, A7-3, A7-5) with Chi² values ranging from 0.312 to 2.85 (P=0.11 to 0.59). A fourth screening population (A7-4) segregated the resistance gene at a higher ratio with a 5% probability (Chi²=4.86) to fit the 3:1 hypothesis.

In contrast to line A, a complex T-DNA insertion pattern was found in line E suggesting the presence of at least 2 T-DNAs. One hygromycin resistant T1 individual of this line, E3, was crossed as the female parent to line CS10 of Arabidopsis thaliana (ecotype Landsberg) as described before. This cross yielded F1 offspring E3-1 to E3-5. As above, F2 screening populations were obtained after self-fertilisation from each F1 plant In these F2 populations the hygromycin resistance -gene segregated at ratios of 3:1 (E3-4, Chi²=0.439; E3-5, Chi²=0.595) and 15:1 (E3-3, Chi²=1.057), respectively, confirming the presence of two segregating T-DNA insertions in line E. Transgene segregation in another F2 screening population (E3-2) did not fit with either segregation hypothesis, but was close to 6:1 (Chi²=0.219).

### Example 7

Effect of (Short) P_{DMC1}::Rad51::nosT on Melotic Recombination in Arabidopsis thaliana

As described in Example 5, individuals (A7 and E3) of two transgenic lines (A and E) containing the (short) P_{DMC1}::Rad51::nosT gene were crossed as female to a non-transgenic line of Arabidopsis thaliana, CS10. The latter line is homozygous for two closely linked (ca. 5 cM) recessive mutations on chromosome 5 which give rise to distinct visual phenotypes. One mutation (tz-201) disrupts when homozygous the synthesis of thiamine leading to production of bleached true leaves if grown without thiamine supplement. The other mutation (cer3-1) affects when homozygous the wax deposition on the surface of epidermal cells. Its phenotype is recognised easily by appearance of glossy inflorescence stems and seed pods; later in development it also affects male fertility if plants are grown at low humidity. F1 plants derived from the crosses (above) to lines A7 and E3 were heterozygous for both visual markers and therefore were phenotypically wildtype (TZ/tz, CER/cer). These plants were also fully fertile and produced F2 progeny after self-fertilisation. All progeny of a given F1 plant were pooled into a single F2 screening population named A7-2 to A7-5 or E3-1 to E3-5, respectively. Due to free combination of parental chromosomes at fertilisation 25% homozygous tz phenotypes are expected in the F2 generation when seeds are germinated on MS medium without thiamine addition. Such segregation ratios for tz were indeed found in F2 screening populations derived for example from A7-4 (Chi²=0.155, P=0.7), A7-5 (Chi²=0.0024, P=0.96) or E3-5 (Chi²=0.202, P=0.66). This indicates that in the corresponding F1 parent the (short) P_{DMC1}::Rad51::nosT transgene did not adversely affect normal chromosome segregation at meiosis.

Thiamine requiring F2 plants (tz/tz) were then transferred to soil and were regularly watered with a 1% thiamine solution to allow for plant recovery and growth. Emerging inflorescences were analysed for cer phenotype. Since the cer mutation is linked to tz, most tz/tz plants were also expected to be homozygous for cer. This linkage is broken only by rare recombination events. The frequency of such events usually is proportional to the distance between the markers. For example, the distance between tz and cer is approximately 5 cM, ie. only 4-5 recombinants are normally expected among 100 F2 plants. Indeed, an average recombination frequency of 5.1% was found between tz and cer in control F2 screening populations which were derived from a control cross of untransformed ecotype C24 to line CS10. However, compared to the controls, the recombination frequency was significantly elevated in screening populations derived from A7×CS10 crosses and from E3×CS10 crosses, respectively (Table 2).

Table 2 summarises the results from the experiment: analysis for Meiotic Recombination Events in Aiabidopsis F1 hybrids. Arabidopsis thaliana (ecotype C24) was transformed with plasmid p3243 comprising within its T-DNA the P_{DMC1}::RAD51::nosT gene. Two transformed lines, A and E, were selected for further analysis. Hygromycin resistant T2 individuals of both lines, A7 and E3, were crossed to an untransformed line of Landsberg ecotype, CS10, which was homozygous for cer and tz. Several F1 progenies from each cross were allowed to self-fertilise and to set seed, giving rise to corresponding F2 screening populations (A7-2 to A7-5, and E3-1 to E3-5). A control cross between untransformed C24 wildtype and CS10 yielded four control F2 screening populations. The screening started with the germination of F2 seed on MS synthetic growth medium as described by Valvekens et al (1988, PNAS 85, 5536-5540) but lacking thiamine. Once identified, thiamine deficient plantlets were transferred to soil to allow for plant recovery and development in the presence of added thiamine. After flowering, the soil grown tz-201/tz-201 plants were scored for presence or absence of the cer phenotype. Recombination events are indicated by CER wildtype phenotype. The table shows the number of tz-201/tz-201 plants transferred to soil, the number of transferred tz-201/tz-20plants in which the linkage to cer is maintained, and the number of transferred tz-201/tz-201 plants in which the linkage to cer is broken by meiotic recombination in the F1 parent generation.

These data demonstrate that meiotic expression of a polynucleotide encoding AtRAD51 protein increases the recombination frequency at F1 meiosis. On average, the recombination frequency in screening populations derived from crosses A7×CS10 and E3×CS10 was increased 2.0-fold and 1.8-fold, respectively, compared to the averaged recombination frequency found in the control experiments.

These results are unexpected given the presumed preference of plant RAD51 for recombination between sister chromatids over recombination between chromosome homologues. Such preference is well documented for yeast RAD51 (J. E. Haber, 2000, Trends in Genetics 16, 259-264). The increase in interhomologue recombination observed above might be due to either of the following: (i) the plant RAD51 does not show the same substrate preference as its yeast homologue. This hypothesis is most unlikely given the high conservation of RAD51 proteins amongst yeast and plants. (ii) the plant RAD51 mimics the substrate preference of yeast RAD51. Since in yeast this preference is not absolute, it is possible that meiotic overexpression of plant RAD51 increases (to different extent) both intersister and interhomologue recombination at meiosis, whereby only the latter is recorded in the experiments above. (iii) RAD51 and DMC1 proteins are co-localised on meiotic chromosome cores and synaptonemal complexes in mouse and lily, possibly indicating direct physical interaction between the two proteins (M. Terasawa et al., 1995, Genes and Development 9, 925-934; M. Tarsounas et al., 1999, J. Cell Biology 147, 207-220). It is thus conceivable that meiotic overexpression of RAD51 protein provides additional substrates or nucleation sites for DMC1 protein thus promoting interhomologue recombination. According to this hypothesis, the availability of RAD51 protein at meiosis is limiting to interhomologue recombination. (iv) Meiotic expression of the RAD51 encoding transgene into RNA alone may be sufficient to promote interhomologue recombination by downregulating the expression of both the endogenous and the foreign RAD51 gene via co-suppression or other mechanisms related to post-transcriptional gene silencing. Gene silencing of this kind often is associated with complex T-DNA insertion patterns such as those found in line E (above). A depletion of RAD51 might then lead to DMC1 taking over the role of RAD51 during meiosis, which in consequence would lead to increased meiotic recombination between homologous/homeologous chromosomes.

### Example 8

Production of Transgenic Corn Expressing Genes Involved in Meiotic Recombination

Since meiotic overexpression of a polynucleotide encoding plant RAD51 protein increases the frequency of meiotic recombination between two markers, it may be used to improve for example corn breeding. A transgenic corn line can be generated which expresses the P_{DMC1}::Rad51::nosT gene following the methods given below.

(a) Method of Transformation

The genetic transformation of corn, whatever the method employed (electroporation, Agrobacterium, microfibers, particle gun) generally requires the use of undifferentiated cells in rapid division which have conserved a capability for regeneration into entire plants. This type of cells is found in embryogenic friable callus (of type II) of corn.

These calli are obtained from immature embryos of Hi II or A188×B73 genotype according to the method and on the media described by Armstrong (in the maize Handbook; 1994, M. Freeling, V. Walbot Eds; pp 665 : 671), and can be maintained and multiplied by transfer (every 15 days) to flesh initiation medium.

Plantlets are regenerated from these calli by modifying the hormonal and osmotic equilibrium of cells as described by Vain et al. (1989, Plant Cell Tissue and Organ Culture 18 : 143-151). These plants are then acclimatised in a greenhouse where they can be crossed or self-fertilised.

A method of genetic transformation leading to the stable integration of the modified genes into the genome of the plant is used. This method is based on the use of a particle gun. The target cells are fragments of calli of a surface area of 10 to 20 mm². They are arranged, 4 h before bombardment, at a rate of 16 fragments per dish, at the centre of a petri dish containing a culture medium identical to the initiation medium, to which 0.2 M mannitol+0.2 M sorbitol are added. The tissues are then bombarded as described previously.

Another method is based on the direct bombardment of immature embryos (10 days after pollination) with plasmid DNA coated on gold particle. They are arranged, 4 h before bombardment, at a rate of 36 fragments per dish, at the centre of a petri dish containing a culture medium identical to the initiation medium, to which 0.2 M mannitol+0.2 M sorbitol are added. The tissues are then bombarded as described previously.

The dishes of calli or embryos bombarded in this way are then sealed with “Scello-frais”, and are cultured in the dark at 27° C. The bombarded tissue is then transferred to initiation medium containing a selective agent whose nature and concentration can vary according to the selective marker used. The first tissue transfer takes place 24 h after bombardment, thereafter once every 15 days for the following 3 months. The selective agents used are generally active compounds of herbicides (Basta®, Round Up®) or antibiotics (hygromycin, kanamycin).

Calli whose growth is not inhibited by the selective agent appear after 3 months or sometimes earlier, each being usually derived from a single cell in which one or more copies of the selection gene has been integrated. The frequency of calli transformation is about 0.8 callus per bombarded dish. These calli are, individualised, identified and cultured to regenerate plants.

In order to avoid contamination with non-transformed cells, all these operations are performed on culture media containing the selective agent. Plants that have been regenerated are acclimatised then cultured in a greenhouse where they can be crossed or self-fertilised.

(b) Use of the Bar Gene for Selection

The bar gene of Streptomyces hygroscopicus encodes for a phosphinotricine acyl transferase (PAT), which inactivates by acetylation the phosphinotricine active molecule of the basta herbicide®. Therefore, cells bearing this gene are rendered resistant to this herbicide and can be selected for.

As far as the transformation of cereals is concerned, the coding sequence of the bar gene is under the control of a regulatory region allowing a high and constitutive expression in plant cells. Such a region may comprise the promoter and the first intron of the actin gene of rice as described by McElroy (1991, Mol. Gen. Genet. 231 : 150-160).

This chimeric gene is cloned in a plasmid allowing its amplification in an Escherichia coli. This plasmid pDM302 (Cao 1992, Plant Cell Report 11 : 586-591), after amplification and purification on a Qiagen column®, can be used for genetic transformation of corn with for example the method described above. In this case 2 mg/l of phosphinotricine are added to the culture media destined for transformed cells selection.

For the introduction of the construct carrying the P_{DMC1}::Rad51::nosT gene, a so-called technique of co-transformation can advantageously be used. A co-precipitation of the two plasmids is carried out (one carrying the gene of the selection and the other carrying the P_{DMC1}::Rad51::nosT gene) on the tungsten or gold particles, the total quantity of DNA precipitated on the particles remaining identical to that which is in the standard protocol (5 μg of DNA per 2.5 mg of particles); each plasmid represents approximately half of the total DNA used.

The experiment shows that with this method the co-integration of the plasmids into plant cells is achieved at high frequency (ca. 90%). Almost every plant, which has the Bar gene integrated into its genome and therefore is resistant to Basta, also carries the P_{DMC1}::Rad51::nosT gene. The percentage of selected plants expressing the P_{DMC1}::Rad51::nosT gene is approximately 70%.

Because the genes thus introduced are genetically linked to the selective marker (Bar), meiotic segregation of the P_{DMC1}::Rad51::nosT gene can conveniently be recognised in the progenies of a transformed plant, thus allowing for identification of homozygous or heterozygous parent plants. These can be used in crosses to other lines for the purpose of increasing meiotic recombination at F1 meiosis between two parental genomes. The resulting recombination events can be identified among the F2 progenies. If the parent plant is heterozygous for the P_{DMC1}::Rad51::nosT gene, the latter will not be transmitted to all F1 offspring. Therefore, F1 offspring which contain the P_{DMC1}::Rad51::nosT gene preferably are identified by using for example the linked selective marker. This identification usually is not required if the parent plant is homozygous for the P_{DMC1}::Rad51::nosT gene, because the transgene will be transmitted to all F1 offspring.

Often, it may be desirable to remove, for example by chromosome segregation, the P_{DMC1}::Rad51::nosT gene from the genome of the plant in which a recombination event has been identified in order to restore a normal recombination behaviour in that plant and/or its progeny. In cases where the P_{DMC1}::Rad51::nosT gene has not already segregated from the genome of the F2 plant, the removal of P_{DMC1}::Rad51::nosT can be achieved for example by outcrossing of the identified F2 plant to another plant not containing the P_{DMC1}::Rad51::nosT gene and by screening the progeny from this cross for individuals which have retained the recombination event but have lost the P_{DMC1}::Rad51::nosT gene. Such individuals can conveniently be recognised in that they have also lost the selection marker.

### Example 9

Production of Maize Transformants and Analysis

Transformations were performed with the following plasmids:

p2034: proDMC1 (short)Rad51-3′nos

p2042: RB-pro nos-NPTII-3′nos+pro DMC1(short)-GUS-3′nos-LB

p3277: RB-pro nos-NPTII_{—}3′nos+pro DMC1(long)-GUS-3′nos-LB

p4928: RB-pro Nos-NPTII-3′nos+proDMC1(long)-Rad51-3′Nos-LB

For each plasmid 200 to 600 immature embryos were bombarded along with the plasmid pDM302 containing the bar gene as selective marker under the control of the rice actin promoter plus its first Intron.

Primary transformants produced in vitro and acclimatised in a growthchamber were crossed in a greenhouse with a maize Elite line.

T1 seeds were harvested for each construct:

p2034 : 25 T0 lines giving T1 seeds lots

p2042 : 30 T0 lines giving T1 seeds lots

p3277 : 21 T0 lines giving T1 seeds lots

p4928 : 54 T0 lines giving T1 seeds lots

Functionality of the DMC1 promoters is assessed by analysis of GUS expression on several T1 plants (plasmids p2042 and p3277) which show resistance to the Basta herbicide. Immature male and female inflorescences at the meiotic stage are subjected to the histochemical GUS staining assay.

Analysis of the effect on frequency of míotic recombination is conducted on progenies of T1 basta resistant plants (plasmids p2034 and p4928)—determined by a leaf painting assay—obtained after back crossing with a line genetically different from the ones used for the transformation procedure and different from the elite line used for pollinating the primary transformants. Reciproqual crosses are done in order to address in the corresponding T2 progenies specifically the recombination effects on male and female meïosis separately. The frequency of genetic recombination is monitored by scoring different polymorphic microsatellites markers on the maize chromosomes. This survey is performed on at least 100 siblings for two to three “T2” progenies per selected transformant in each cross.

TABLE 1
Cytotoxicity of AtRAD51 expression in E. coli

| | REC A + [cfu/ml] | | | REC A − [cfu/ml] | | |
|---|---|---|---|---|---|---|
| CONSTRUCT | −IPTG | +IPTG | +IPTG #2 | −IPTG | +IPTG | +IPTG #2 |
| pSE380/control | 0.9 × 10⁹ | 1.1 × 10⁹ | na | 1.6 × 10⁹ | 1.6 × 10⁹ | na |
| | 1.8 × 10⁹ | 1.9 × 10⁹ | na | na | na | na |
| pSE380/AtRAD51 | 7.8 × 10⁸ | 1.1 × 10⁵ | na | 3.6 × 10⁷ | 1.5 × 10⁴ | 4/155 |
| | 1.0 × 10⁹ | 1.8 × 10⁵ | 5/181 | na | na | na |
| pYT11/ | 1.6 × 10⁹ | 1.8 × 10⁹ | na | na | na | na |
| AtRAD51::INTEIN | | | | | | |

TABLE 2
Recombination Analysis on Chromosome 5 between tz-201 - [5 cM] - cer-3

| | | | | | |
|---|---|---|---|---|---|
| | | | | | Increased |
| | | | | | Recombination |
| Line/ | | | | % CER/tz-201 | (relative to WT |
| F2 population | cer/tz-201 | CER/tz-201 | total tz-201 | (of Total tz-201) | control |

pDMC1(short)::Rad 51::nosT crossed to CS10

| | | | | | |
|---|---|---|---|---|---|
| A7/2 | 218 | 26 | 242 | 10.7 | |
| 3 | 228 | 20 | 248 | 8.1 | |
| 4 | 239 | 26 | 262 | 9.9 | |
| 5 | 190 | 27 | 217 | 12.4 | |
| | | | | average: 10.3 | 2.0 |
| E3/1 | 202 | 19 | 221 | 8.6 | |
| 2 | 228 | 17 | 245 | 6.9 | |
| 3 | 258 | 31 | 289 | 10.7 | |
| 4 | 262 | 31 | 293 | 10.6 | |
| 5 | 234 | 26 | 260 | 10.0 | |
| | | | | average: 9.4 | 1.8 |

Controls: (untransformed) C24 crossed to CS10

| | | | | | |
|---|---|---|---|---|---|
| CTL/1 | 300 | 12 | 312 | 3.8 | |
| CTL/2 | 149 | 9 | 158 | 5.7 | |
| CTL/3 | 178 | 15 | 193 | 7.8 | |
| CTL/4 | 159 | 5 | 164 | 3.0 | |
| | | | | average: 5.1 | 1.0 |

1. An expression cassette comprising a meiotically active promoter operably linked to a polynucleotide encoding a recombinational DNA repair polypeptide, or fragment thereof, wherein said polynucleotide is capable of stimulating plant meiotic recombination when expressed into RNA and/or said polypeptide.

2. The expression cassette of claim 1, wherein said polynucleotide capable of stimulating meiotic recombination in plants encodes a recombinational DNA repair polypeptide, or a fragment thereof, selected from the group consisting of: SPO11 (protein ID AAA65532.1), MER1 (protein ID NP_{—}014189), MER2 (protein ID AAA34772.1), MRE2 (protein ID BAA02016.1), MEI4 (protein ID NP_{—}010963.1), REC102 (protein ID AAA34964.1), REC104 (protein ID AAB26085.1), REC114 (protein IDNP_{—}013852.1), MRE11 (protein ID BAA02017.1), XRS2 (protein ID AAA35220.1), RAD18 (SMC) (protein ID AAA34932.1), RAD50 (protein ID CAA32919.1), RAD51 (protein ID BAA00913.1, protein ID CAA45563, protein ID AAB37762.l, protein ID AAD32030.1, protein ID AAD32029.1, AAC23700 or AAF69145.1), RAD52 (protein ID AAA50352.1), RAD54 (protein ID AAA34949.1), RDH54/TID1 (protein ID NP_{—}009629), RAD55-57 (protein ID protein ID AAA19688.1, protein ID AAA34950.1), DMC1 (protein ID NP_{—}011106.1), and Arabidopsis protein XRS9, or functional fragments or analogues thereof (wherein the protein ID provides a cross-reference to GenBank for the corresponding nucleic acid sequence encoding the relevant polypeptide).

3. The expression cassette of claim 1, wherein said polynucleotide encodes a RAD51 polypeptide, or a fragment thereof.

4. The expression cassette of claim 3, wherein said polypeptide is a plant RAD51 polypeptide, or a fragment thereof.

5. The expression cassette of claim 4, wherein said polypeptide is selected from the group consisting of: Arabidopsis thaliana RAD51 represented by protein ID AAB37762.1 (AtRAD51), or a fragment thereof; Zea mais RAD51 represented by protein ID AAD32029.1 (ZmRAD51A) or a fragment thereof, or protein ID AAD32030.1 (ZmRAD51B), or a fragment thereof; and tomato RAD51 polypeptide represented by protein ID No AAC23700 (LeRAD51), or a fragment thereof.

6. The expression cassette of any one of claims 1-5, wherein said meiotically active promoter is a meiosis specific promoter.

7. The expression cassette of claim 6, wherein the promoter is active during zygotene and pachytene of meiosis I.

8. The expression cassette of claim 7, wherein the promoter is a plant DMC1 promoter.

9. The expression cassette of claim 8, wherein the promoter is a plant DMC1 short promoter.

10. The expression cassette of claim 8, wherein the promoter is a plant DMC1 long promoter.

11. A recombinant vector comprising the expression cassette of any one of claims 1-10.

12. A host cell transformed with the expression cassette of any one of claims 1-10, or the vector of claim 11.

13. The host cell of claim 12, wherein said host cell is a plant cell.

14. The host cell of claim 13, wherein said plant cell is selected from any one of the following tissues: leaf, root, seed, stem or flower-tissues.

15. The host cell of claim 13 or 14, wherein said plant cell selected from the group of plants consisting of members of the following families: Cruciferae, Umbelliferae, Gramineae, Solanaceae, Compositae, Malvaceae, Leguminosae and Cucurbitaceae.

16. The host cell of claim 15, wherein said plant is selected from the following crops: oil seed rape, cauliflower broccoli; carrot, maize, wheat and barley, tomato, potato, tobacco, sunflower, cotton, soybean, pea and melon.

17. A plant comprising the host cell of any one of claims 12-16.

18. A plant transformed or transfected with the expression cassette of any one of claims 1-10, or the vector of claim 11.

19. Seed of the plant of claim 17 or 18.

20. A method for increasing the frequency of homologous or homeologous recombination in a plant, wherein said method comprises
a) transforming or transfecting a plant cell or tissue with a polynucleotide encoding a recombinational DNA repair polypeptide, or a fragment thereof, wherein said polynucleotide is capable of stimulating plant meiotic recombination when expressed into RNA and/or said polypeptide, or said polynucleotide is capable of stimulating plant meiotic recombination when introduced into said plant cell or tissue as an RNA::DNA chimeric molecule;
b) culturing said transformed or transfected plant cell or tissue under conditions allowing the regeneration of a plant,
c) culturing said regenerated plant under conditions allowing sexual reproduction of said regenerated plant; and
d) expressing said polynucleotide in said regenerated plant;
e) obtaining a sexually reproduced plant which is the product of said sexual reproduction; and
f) screening said sexually reproduced plant and/or its progeny for homologous or homeologous recombination events.

21. A method for increasing the frequency of homologous or homeologous recombination in a plant, wherein said method comprises:
a) transforming or transfecting a plant cell or tissue with an expression cassette in accordance with any one of claims 1-10, or the vector of claim 11;
b) culturing said transformed or transfected plant cell or tissue under conditions allowing the regeneration of a plant,
c) culturing said regenerated plant under conditions allowing sexual reproduction of said regenerated plant; and
d) expressing said polynucleotide in said regenerated plant;
e) obtaining a sexually reproduced plant which is the product of said sexual reproduction; and
f) screening said sexually reproduced plant and/or its progeny for homologous or homeologous recombination events.

22. The method of claim 20 or 21, wherein the plant regenerated from the transformed or transfected plant cell or tissue is:
a) crossed with a plant from a second plant line, to generate a hybrid plant,
b) expressing said polynucleotide in said hybrid plant;
c) culturing said regenerated plant under conditions allowing sexual reproduction of said hybrid plant; and
d) screening progeny of said hybrid plant for homologous or homeologous recombination events.

23. A method for increasing the frequency of homologous or homeologous meiotic recombination in a plant cell capable of undergoing meiosis, wherein said method comprises transforming or transfecting said plant cell with a polynucleotide encoding a recombinational DNA repair polypeptide, or a fragment thereof wherein said polynucleotide is capable of stimulating meiotic recombination when expressed into RNA and/or said polypeptide, or said polynucleotide is capable of stimulating meiotic recombination when introduced into a plant cell as an RNA::DNA chimeric molecule.

24. A method for increasing the frequency of homologous or homeologous meiotic recombination in a plant cell capable of undergoing meiosis, wherein said method comprises transforming or transfecting said plant cell with the expression cassette of any one of claims 1-10, or the vector of claim 11.

25. The method of claim 23 or 24, wherein the plant cell is a meiocyte.

26. The method of any one of claims 23 to 25, wherein said method further comprises culturing the transformed plant cell under conditions permitting regeneration of a fertile plant.

27. The method of claim 26, wherein said method further comprises:
a) obtaining a hybrid between the fertile plant (first parent line) and a second parent line, or cells thereof;
b) expressing said polynucleotide in said hybrid plant;
c) permitting said hybrid plant to self-fertilise and produce offspring plants; and
d) screening progeny of said hybrid plant for homologous or homeologous recombination events.

28. A method for obtaining a plant having a desired characteristic, wherein said method comprises:
a) transforming or transfecting a plant cell or tissue with a polynucleotide encoding a recombinational DNA repair polypeptide, or a fragment thereof wherein said polynucleotide is capable of stimulating plant meiotic recombination when expressed into RNA and/or said polypeptide, or said polynucleotide is capable of simulating plant meiotic recombination when introduced into said plant cell or tissue as an RNA::DNA chimeric molecule;
b) culturing said transformed or transfected plant cell or tissue under conditions allowing the regeneration of a plant,
c) permitting said regenerated plant to self-fertilise to produce a first parent line;
d) obtaining a hybrid between a plant of the first parent line and a second parent line, or cells thereof;
e) expressing said polynucleotide in said hybrid plant;
f) permitting said hybrid plant to self-fertilise and produce offspring plants; and
g) screening said offspring plants for plants having said desired characteristic.

29. A method for obtaining a plant having a desired characteristic, wherein said method comprises:
a) transforming or transfecting a plant cell or tissue with an expression cassette in accordance with any one of claims 1-10, or the vector of claim 11;
b) culturing said transformed or transfected plant cell or tissue under conditions allowing the regeneration of a plant,
c) permitting said regenerated plant to self-fertilise to produce a first parent line;
d) obtaining a hybrid between a plant of the first parent line and a second parent line, or cells thereof;
e) expressing said polynucleotide in said hybrid plant;
f) permitting said hybrid plant to self-fertilise and produce offspring plants; and
g) screening said offspring plants for plants having said desired characteristic.

30. The method of any one of claims 20 to 29, wherein said polynucleotide capable of stimulating meiotic recombination in plants encodes a recombinational DNA repair polypeptide, or a fragment thereof, selected from the group consisting of: SPO11 (protein ID AAA65532.1), MER1 (NP_{—}014189, MER2 (protein ID AAA3477-2.1), MRE2 (protein ID BAA02016.1), MEI4 (protein ID NP_{—}010963.1), REC102 (protein ID AAA34964.1), REC104 (protein ID AAB26085.1), REC114 (protein ID NP_{—}013852.1), MRE11 (protein ID BAA02017.1), XRS2 (protein ID AAA352-20.1), RAD18 (SMC) (protein ID AAA34932.1), RAD50 (protein ID CAA32919.1), RAD51 (protein ID BAA00913.1, AAB37762.1, AAD32030.1, AAD32029.1, AAC23700 or AAF69145.1), RAD52 (protein ID AAA50352.1), RAD54 (protein ID AAA34949.1), RDH54/TID1 (protein ID NP_{—}009629), RAD55-57 (protein ID AAA19688.1, AAA34950.1), DMC1 (protein ID NP_{—}011106.1), and Arabidopsis protein XRS9, or functional fragments or analogues thereof.

31. A plant produced in accordance with the method of any one of claims 20 to 30.

32. Seed from the plant of claim 31.

33. Use of a plant of any one of claims 17, 18 or 31 for plant breeding.
